(19) United States
(12) Patent Application Publication
(10) Pub. No.: US 2023/0127345 A1
(43) Pub. Date: Apr. 27, 2023

(54) SELENOGALACTOSIDE COMPOUNDS FOR THE PREVENTION AND TREATMENT OF DISEASES ASSOCIATED WITH GALECTIN AND THE USE THEREOF

(71) Applicant: GALECTIN SCIENCES, LLC, Norcross, GA (US)

(72) Inventors: Sharon Shechter, Andover, MA (US); Eliezer Zomer, Newton, MA (US); Peter G. Traber, Alpharetta, GA (US); Raphael Nir, Needham, MA (US); Joseph M. Johnson, Arlington, MA (US); Ryan George, Framingham, MA (US)

(21) Appl. No.: 18/052,814

(22) Filed: Nov. 4, 2022

Related U.S. Application Data
(63) Continuation of application No. 16/080,423, filed on Aug. 28, 2018, now abandoned.
(60) Provisional application No. 62/303,872, filed on Mar. 4, 2016.

(51) Int. Cl. C07H 19/056; A61P 1/16; A61P 9/00; A61P 13/12; A61P 11/00; A61K 31/7056; A61K 45/06

(52) U.S. Cl. CPC C07H 19/056; A61P 1/16; A61P 9/00; A61P 13/12; A61P 11/00; A61K 31/7056; A61K 45/06

(57) ABSTRACT

Aspects of the invention relate to novel synthetic compounds having a binding affinity with galectin proteins.

### RELATED APPLICATION(S)

This application is a continuation of U.S. application Ser. No. 16/080,423, filed Aug. 28, 2018, which is a U.S. national phase application under 35 U.S.C. 371 of PCT International Application No. PCT/US2017/020658, which claims the benefit of and priority to U.S. Provisional Application Ser. No. 62/303,872, filed Mar. 4, 2016, the entire disclosure of each of which is incorporated herein by reference in their entireties.

### FIELD OF THE INVENTION

Aspects of the invention relate to compounds, pharmaceutical compositions, methods for the manufacturing of compounds and methods for treatment of various disorders mediated at least in part by one or more galectins.

### BACKGROUND OF THE INVENTION

Galectins are a family of S-type lectins that bind beta-galactose-containing glycoconjugates. To date, fifteen mammalian galectins have been isolated. Galectins regulate different biological processes such as cell adhesion, regulation of growth, apoptosis, inflammation, fibrogenesis, tumor development and progression. Galectins have been shown to be involved in inflammation, fibrosis formation, cell adhesion, cell proliferation, metastasis formation, angiogenesis, cancer and immunosuppression.

### SUMMARY OF THE INVENTION

Aspects of the invention relate to compounds or compositions comprising a compound in an acceptable pharmaceutical carrier for parenteral or enteral administration, for use in therapeutic formulations. In some embodiments, the composition can be administered parenterally via an intravenous, subcutaneous, or oral route.

Aspects of the invention relate to compounds or compositions for the treatment of various disorders in which lectin proteins play a role in the pathogenesis, including but not limited to, chronic inflammatory diseases, fibrotic diseases, and cancer. In some embodiments, the compound is capable of mimicking glycoprotein interactions with lectins or galectin proteins which are known to modulate the pathophysiological pathways leading to immune recognition, inflammation, fibrogenesis, angiogenesis, cancer progression and metastasis.

In some embodiments, the compound comprises pyranosyl and/or furanosyl structures bound to a selenium atom on the anomeric carbon of the pyranosyl and/or furanosyl.

In some embodiments, specific aromatic substitutions can be added to the galactose core or heteroglycoside core to further enhance the affinity of the selenium bound pyranosyl and/or furanosyl structures. Such aromatic substitutions can enhance the interaction of the compound with amino acid residues (e.g. Arginine, Tryptophan, Histidine, Glutamic acid etc . . . ) composing the carbohydrate-recognition-domains (CRD) of the lectins and thus strengthen the association and binding specificity.

In some embodiments, the compound comprises monosaccharides, disaccharides and oligosaccharides of galactose or a heteroglycoside core bound to a selenium atom (Se) on the anomeric carbon of the galactose or of the heteroglycoside.

In some embodiments, the compound is a symmetric digalactoside wherein the two galactosides are bound by one or more selenium bonds. In some embodiments, the compound is a symmetric digalactoside wherein the two galactosides are bound by one or more selenium bonds and wherein the selenium is bound to the anomeric carbon of the galactose. In some embodiments, the compound is a symmetric digalactoside wherein the two galactosides are bound by one or more selenium bonds and one or more sulfur bonds and wherein the selenium is bound to the anomeric carbon of the galactose. Yet in other embodiments, the compound can be an asymmetric digalactoside. For example, the compound can have different aromatic or aliphatic substitutions on the galactose core.

In some embodiments, the compound is a symmetric galactoside having one or more selenium on the anomeric carbon of the galactose. In some embodiments, the galactoside has one or more selenium bound to the anomeric carbon of the galactose and one or more sulfur bound to the selenium. In some embodiments, the compound can have different aromatic or aliphatic substitutions on the galactose core.

Without being bound to the theory, it is believed that the compounds containing the Se containing molecules render the compound metabolically stable while maintaining the chemical, physical and allosteric characteristics for specific interaction with lectins or galectins known to recognize carbohydrates.

In some embodiments, the monogalactoside, digalactoside or oligosaccharides of galactose of the present invention are metabolically more stable than compounds having an O-glycosidic or S-glycosidic bond.

In some embodiments, the compound is a monomeric-selenium polyhydroxylated- cycloalkanes compound having Formula (1) or Formula (2) or a pharmaceutically acceptable salt or solvate thereof:

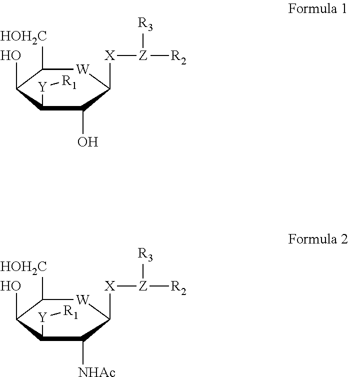

### Wherein X is Selenium;

Wherein Z is a carbohydrate or linkage consisting of O, S, C, NH, CH2, Se, amino acid to R₂ and R₃;
Wherein W is selected from the group consisting of O, N, S, CH2, NH, and Se;
Wherein Y is selected from the group consisting of O, S, C, NH, CH2, Se, amino acid, and a combination thereof.
Wherein R₁, R₂, and R₃ are independently selected from the group consisting of CO, SO2, SO, PO2, PO, CH, Hydrogen, hydrophobic linear and cyclic hydrocarbons including heterocyclic substitutions of molecular weight of about 50-200 D.

In some embodiments, the hydrophobic linear and cyclic hydrocarbons can comprise one of : a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, or a substituted imino group

In some embodiments, the compound is a dimeric-polyhydroxylated-cycloalkane compound.

In some embodiments, the compound has the general Formula (3) or Formula (4) or a pharmaceutically acceptable salt or solvate thereof:

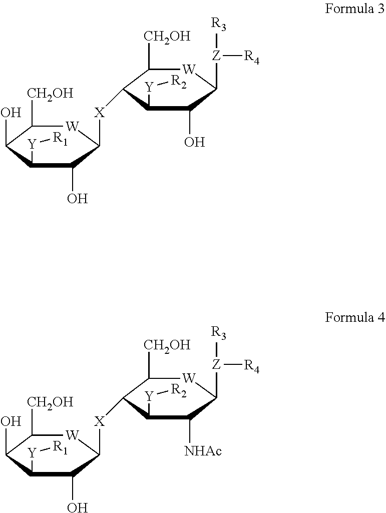

### Wherein X is Se, Se—Se or Se—S;

Wherein Z is independently selected from a carbohydrate (composing, for example, an oligomeric Se-galactoside) or linkage consisting of O, S, C, NH, CH2, Se, and amino acid to R₃ and R₄;
Wherein W is selected from the group consisting of O, N, S, CH2, NH, and Se;
Wherein Y is selected from the group consisting of O, S, C, NH, CH2, Se, and amino acid;
Wherein R₁, R₂, R₃, and R₄ are independently selected from the group consisting of CO, SO2, SO, PO2, PO, CH, Hydrogen, and hydrophobic linear and cyclic hydrocarbons including heterocyclic substitutions of molecular weight of about 50-200 D.

In some embodiments, the hydrophobic linear and cyclic hydrocarbons can comprise one of: a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, or a substituted imino group.

In some embodiments, the compound is a 3-derivatized diselenogalactoside bearing a fluorophenyl-triazole.

Aspect the present invention relates to a compound of formula (5) or a pharmaceutically acceptable salt or solvate thereof:

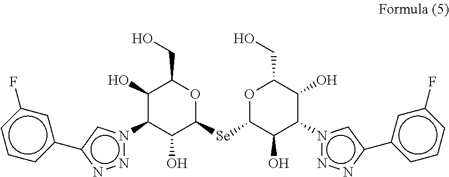

Aspect the present invention relates to a compound of formula (6) or formula (7) or a pharmaceutically acceptable salt or solvate thereof:

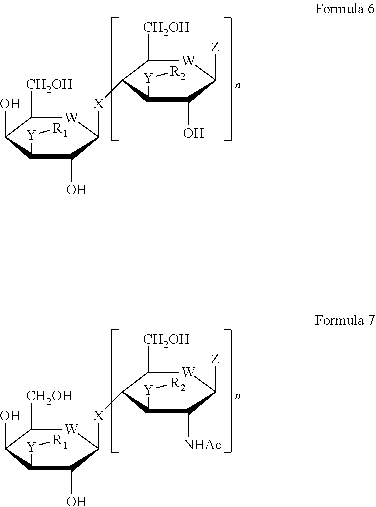

Wherein n≤24;

### Wherein X is Se, Se—Se or Se—S;

Wherein W is selected from the group consisting of O, N, S, CH2, NH, and Se;
Wherein Y, and Z are independently selected from the group consisting of O, S, C, NH, CH2, Se, and amino acid;
Wherein R₁ and R₂ are independently selected from the group consisting of CO, SO2, SO, PO2, PO, CH, Hydrogen, hydrophobic linear and cyclic hydrocarbon including heterocyclic substitutions of molecular weight of 50-200 D including, but not limited to:
a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens;
b) a phenyl group substituted with at least one car boxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group; and
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted With at least one substituted carbonyl group,
e) a saccharide; a substituted saccharide; D-galactose; substituted D-galactose; C3-[1,2,3]-triaZol-1-yl-substituted D-galactose; hydrogen, an alkyl group, an alkenyl group, an aryl group, a heteroaryl group, and a heterocycle and derivatives; an amino group, a substituted amino group, an imino group, or a substituted imino group.

In some embodiments, the compound is in a free form. In some embodiments, the free form is an anhydrate. In some embodiments, the free form is a solvate, such as a hydrate.

In some embodiments, the compound is in a crystalline form.

Some aspects of the present invention relate to a compound of the invention for use as a therapeutic agent in a mammal, such as a human. In some embodiments, the compound has the formula (1), (2), (3), (4), (5), (6) or (7) and can be used as a therapeutic agent in a mammal, such as a human.

Some aspects of the present invention relate to a pharmaceutical composition comprising the compound of the invention and optionally a pharmaceutically acceptable additive, such as carrier or excipient. In some embodiments, the pharmaceutical composition comprising the compound of formulae (1), (2), (3), (4), (5), (6) or (7) or a pharmaceutically acceptable salt or solvate thereof and optionally a pharmaceutically acceptable additive, such as carrier or excipient.

In some embodiments, the compounds of the present invention bind to one or more galectins. In some embodiments, the compound binds to Galectin-3, Galectin-1, Galectin 8, and/or Galectin 9.

In some embodiments, the compounds of the present invention have high selectivity and affinity for Galectin-3. In some embodiments, the compounds of the present invention have an affinity of about 1 nM to about 50 μM for Galectin-3.

Aspects of the invention relate to compositions or compounds that can be used in the treatment of diseases. Aspects of the invention relate to compositions or compounds that can be used in the treatment of diseases in which galectins are at least in part involved in the pathogenesis. Other aspects of the invention relate to methods of treatment of a disease in a subject in need thereof.

In some embodiments, the composition or the compound can be used in the treatment of nonalcoholic steatohepatitis with or without liver fibrosis, inflammatory and autoimmune disorders, neoplastic conditions or cancers.

In some embodiments, the composition can be used in the treatment of liver fibrosis, kidney fibrosis, lung fibrosis, or heart fibrosis.

In some embodiments, the composition or the compound is capable of enhancing anti-fibrosis activity in organs, including but not limited to, liver, kidney, lung, and heart.

In some embodiments, the composition or the compound can be used in treatment of inflammatory disorders of the vasculature including atherosclerosis and pulmonary hypertension.

In some embodiments, the composition or the compound can be used in the treatment of heart disorders including heart failure, arrhythmias, and uremic cardiomyopathy.

In some embodiments, the composition or the compound can be used in the treatment of kidney diseases including glomerulopathies and interstitial nephritis.

In some embodiments, the composition or the compound can be used in the treatment of inflammatory, proliferative and fibrotic skin disorders including but not limited to psoriasis and scleroderma.

Aspects of the invention relates to methods of treating allergic or atopic conditions, including but not limited to eczema, atopic dermatitis, or asthma.

Aspects of the invention relates to methods of treating inflammatory and fibrotic disorders in which galectins are at least in part involved in the pathogenesis, by enhancing anti-fibrosis activity in organs, including but not limited to liver, kidney, lung, and heart.

Aspects of the invention relates to methods relates to a composition or a compound that has a therapeutic activity to treat nonalcoholic steatohepatitis (NASH). In other aspects, the invention elates to a method to reduce the pathology and disease activity associated with nonalcoholic steatohepatitis (NASH).

Aspects of the invention relates to a composition or a compound used in treating or a method of treating inflammatory and autoimmune disorders in which galectins are at least in part involved in the pathogenesis including but not limited to arthritis, systemic lupus erythematosus, rheumatoid arthritis, asthma, and inflammatory bowel disease.

Aspects of the invention relates to a composition or a compound to treat neoplastic conditions (e.g. benign or malignant neoplastic diseases) in which galectins are at least in part involved in the pathogenesis by inhibiting processes promoted by the increase in galectins. In some embodiments, the invention relates a method of treating neoplastic conditions (e.g. benign or malignant neoplastic diseases) in which galectins are at least in part involved in the pathogenesis by inhibiting processes promoted by the increase in galectins. In some embodiments, the composition or a compound can be used to treat or prevent tumor cell growth, invasion, metastasis, and neovascularization. In some embodiments, the composition or a compound can be used to treat primary and secondary cancers.

Aspects of the invention relates to a composition or a compound to treat neoplastic conditions in combination with other anti-neoplastic drugs including but not limited to checkpoint inhibitors (anti-CTLA2, anti-PD1, anti-PDL1), other immune modifiers including but not limited to anti-OX40, and multiple other anti-neoplastic agents of multiple mechanisms.

In some embodiments, a therapeutically effective amount of the compound or of the composition can be compatible and effective in combination with a therapeutically effective amount of various anti-inflammatory drugs, vitamins, other pharmaceuticals and nutraceuticals drugs or supplement, or combinations thereof without limitation.

Some aspects of the present invention relate to a compound of formula (1), (2), (3), (4), (5), (6) or (7) or a pharmaceutically acceptable salt or solvate thereof for use in a method for treating a disorder relating to the binding of a galectin. Some aspects of the present invention relate to a compound of formulae (1), (2), (3), (4), (5), (6) or (7) or a pharmaceutically acceptable salt or solvate thereof for use in a method for treating a disorder relating to the binding of galectin-3 to a ligand.

Some aspects of the present invention relate to a method for treatment of a disorder relating to the binding of a galectin, such as galectin-3, to a ligand in a human, wherein the method comprises administering a therapeutically effective amount of at least one compound of formulae (1), (2), (3), (4), (5), (6) or (7) or a pharmaceutically acceptable salt or solvate thereof to a human in need thereof.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a high-definition 3D structure of galectin-3 with the CRD sites (Site A, Site B, Site C).

FIG. 2 depicts galectin-3 CRD binding pocket.

FIG. 3 depicts galectin-3 CRD binding pocket with bound galactose units.

FIG. 4 depicts the synthesis of a compound according to some embodiments.

FIG. 5A depicts the inhibition of galectin using a monoclonal antibodies binding assay according to some embodiments.

FIG. 5B depicts the inhibition of galectin using an integrin functional assay according to some embodiments.

FIG. 6A depicts FRET assay (fluorescent resonance energy transfer) assays according to some embodiments.

FIG. 6A depicts a fluorescent polarization assay according to some embodiments.

FIGS. 7A and 7B show the inhibition with the thiogalactoside TD-139 (G-240) and the selenogalactoside G-625 compounds.

FIG. 8A shows the inhibition of Galectin 3 binding with the diselenogalactoside G-626 compound using a fluorescent polarization assay.

FIG. 8B shows Se-monosaccharide (G662) Inhibition of Fluorescent polarization of Galectin-3 binding using a fluorescent polarization assay.

FIGS. 8C and 8D show a hypothetical tetrameric se-galactoside (FIG. 8D) with higher affinity to the Galectin-3 CRD versus the trimeric structure (FIG. 8C) due to additional potential interaction of hydroxyl groups with amino acids in the CRD vicinity thus better inhibition of a fluorescent polarization signal.

FIG. 9 shows the inhibition with the selenogalactoside G-625 compound using an ELISA assay with anti-Galectin-3 antibodies.

FIG. 10 shows the galectin-3 binding inhibition of the thiogalactoside G-240 and the seleno digalactoside G-625 compounds.

FIG. 11A shows the integrin aVB3 inhibition of the thiogalactoside G-240 and the seleno digalactoside G-625 compounds.

FIG. 11B shows the integrin aVB6 inhibition of the thiogalactoside G-240 and the seleno digalactoside G-625 compounds.

FIG. 11C shows the integrin aMB2 inhibition of the thiogalactoside G-240 and the seleno digalactoside G-625 compounds.

FIG. 11D shows the inhibition of Integrin (aMB2) by the Se-monosaccharide G-656.

FIG. 11E shows the inhibition of Integrin (aMB2) by the Se-monosaccharide G-662.

FIG. 12A shows the cell culture viability (MCF-7 cells) of G-625 at concentrations that have physiological effect on inflammation and fibrogenesis in cell culture models.

FIG. 12B shows the cell culture viability (HTB-38) of G-625 at concentrations that have physiological effect on inflammation and fibrogenesis in cell culture models.

FIG. 13A shows the inhibition of the inflammatory bio-marker MCP-1 by G625 in endotoxin stressed THP-1 Monocytes.

FIG. 13B shows the inhibition of the inflammatory bio-marker MCP-1 by and the viability by MTT in presence of G625, G626 and G-240 (TD-139) in endotoxin stressed THP-1 Monocytes.

FIG. 14A shows the total Gal-3 in in TGFb1 Stimulated Hepatic fibrogenesis of Stellate Cells and effect by G-625 and TD-139 using a fluorescent flow cytometric method for the detection of cellular galectin-3.

FIG. 14B shows the inhibition of galectin-3 secretion in TGFb1 Stimulated Hepatic fibrogenesis of Stellate Cells and effect by G-625 and TD-139 using a fluorescent flow cytometric method for the detection of cellular galectin-3.

FIG. 15: shows Inhibition by G625 of Integrin binding with other Galectins (e.g. Galectin 1 and Galectin 9).

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Citation of documents herein is not intended as an admission that any of the documents cited herein is pertinent prior art, or an admission that the cited documents are considered material to the patentability of the claims of the present application.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for additional factors not described, unless the context clearly dictates otherwise. In addition, throughout the specification, the meaning of “a,” “an,” and “the” include plural references.

Unless otherwise specified, all percentages expressed herein are weight/weight.

Aspects of the invention relate to compositions of mono, disaccharides and oligosaccharides of Galactose (or heteroglycoside) core bound to a selenium atom on the anomeric carbon of the Galactose (or heteroglycoside). In some embodiments, the Se containing molecules render them metabolically stable while maintaining the chemical, physical and allosteric characteristics for specific interaction with lectins known to recognize carbohydrates. In yet other embodiments, the specific aromatic substitutions added to the galactose core further enhance the affinity of the Selenium bound pyranosyl and/or furanosyl structures by enhancing their interaction with amino acid residues (e.g. Arginine, Tryptophan, Histidine, Glutamic acid etc . . . ) composing the carbohydrate-recognition-domains (CRD) of the lectins and thus strengthening the association and binding specificity.

### Galectins

Galectins (also known as galaptins or S-lectins) are a family of lectins which bind beta-galactoside. Galectin as a general name was proposed in 1994 for a family of animal lectins (Barondes, S. H., et al.: Galectins: a family of animal beta-galactoside-binding lectins. Cell 76, 597-598, 1994). The family is defined by having at least one characteristic carbohydrate recognition domain (CRD) with an affinity for beta-galactosides and sharing certain sequence elements. Further structural characterization segments the galectins into three subgroups including: (1) galectins having a single CRD, (2) galectins having two CRDs joined by a linker peptide, and (3) a group with one member (galectin-3) which has one CRD joined to a different type of N-terminal domain. The galectin carbohydrate recognition domain is a beta-sandwich of about 135 amino acids. The two sheets are slightly bent with 6 strands forming the concave side, also called the S-face, and 5 strands forming the convex side, the F-face). The concave side forms a groove in which carbohydrate is bound (Leffler H, Carlsson S, Hedlund M, Qian Y, Poirier F (2004). “Introduction to galectins”. Glycoconj. J. 19 (7-9): 433-40).

A wide variety of biological phenomena have been shown to be related to galectins, including development, differentiation, morphogenesis, tumor metastasis, apoptosis, RNA splicing, and many others.

Generally, the carbohydrate domain binds to galactose residues associated with glycoproteins. Galectins show an affinity for galactose residues attached to other organic compounds, such as in lactose [(β-D-Galactosido)-D-glucose], N-acetyl-lactosamine, poly-N-acetyllactosamine, galactomannans, or fragments of pectins. However, it should be noted that galactose by itself does not bind to galectins.

Plant polysaccharides like pectin and modified pectin have been shown to bind to galectin proteins presumably on the basis of containing galactose residues that are presented in the context of a macromolecule, in this case a complex carbohydrate rather than a glycoprotein in the case of animal cells.

At least fifteen mammalian galectin proteins have been identified which have one or two carbohydrate domain in tandem.

Galectin proteins are found in the intracellular space where they have been assigned a number of functions and they are also are secreted into the extracellular space where they have different functions. In the extracellular space, galectin proteins can have multiple functions that are mediated by their interaction with galactose containing glycoproteins including promoting interactions between glycoproteins that may modulate function or, in the case of integral membrane glycoprotein receptors, modification of cellular signaling (Sato et al “Galectins as danger signals in host-pathogen and host-tumor interactions: new members of the growing group of “Alarmins.” In “Galectins,” (Klyosov, et al eds.), John Wiley and Sons, 115-145, 2008, Liu et al “Galectins in acute and chronic inflammation,” Ann. N.Y. Acad. Sci. 1253: 80-91, 2012). Galectin proteins in the extracellular space can additionally promote cell-cell and cell matrix interactions (Wang et al., “Nuclear and cytoplasmic localization of galectin-1 and galectin-3 and their roles in pre-mRNA splicing.” In “Galectins” (Klyosov et al eds.), John Wiley and Sons, 87-95, 2008). In regards to intracellular space, galectin functions appear to be more related to protein-protein interactions, although intracellular vesicle trafficking appears to be related to interaction with glycoproteins.

Galectins have been shown to have domains which promote homodimerization. Thus, galectins are capable of acting as a “molecular glue” between glycoproteins. Galectins are found in multiple cellular compartments, including the nucleus and cytoplasm, and are secreted into the extracellular space where they interact with cell surface and extracellular matrix glycoproteins. The mechanism of molecular interactions can depend on the localization. While galectins can interact with glycoproteins in the extracellular space, the interactions of galectin with other proteins in the intracellular space generally occurs via protein domains. In the extracellular space the association of cell surface receptors may increase or decrease receptor signaling or the ability to interact with ligands.

Galectin proteins are markedly increased in a number of animal and human disease states, including but not limited to diseases associated with inflammation, fibrosis, autoimmunity, and neoplasia. Galectins have been directly implicated in the disease pathogenesis, as described below. For example, diseases states that may be dependent on galectins include, but are not limited to, acute and chronic inflammation, allergic disorders, asthma, dermatitis, autoimmune disease, inflammatory and degenerative arthritis, immune-mediated neurological disease, fibrosis of multiple organs (including but not limited to liver, lung, kidney, pancreas, and heart), inflammatory bowel disease, atherosclerosis, heart failure, ocular inflammatory disease, a large variety of cancers.

In addition to disease states, galectins are important regulatory molecules in modulating the response of immune cells to vaccination, exogenous pathogens and cancer cells.

One of skill in the art will appreciate that compounds that can bind to galectins and/or alter galectin's affinity for glycoproteins, reduce hetero- or homo-typic interactions between galectins, or otherwise alter the function, synthesis, or metabolism of galectin proteins may have important therapeutic effects in galectin-dependent diseases.

Galectin proteins, such as galectin-1 and galectin-3 have been shown to be markedly increased in inflammation, fibrotic disorders, and neoplasia (Ito et al. “Galectin-1 as a potent target for cancer therapy: role in the tumor microenvironment”, Cancer Metastasis Rev. PMID: 22706847 (2012), Nangia-Makker et al. Galectin-3 binding and metastasis,” Methods Mol. Biol. 878: 251-266, 2012, Canesin et al. Galectin-3 expression is associated with bladder cancer progression and clinical outcome,” Tumour Biol. 31: 277-285, 2010, Wanninger et al. “Systemic and hepatic vein galectin-3 are increased in patients with alcoholic liver cirrhosis and negatively correlate with liver function,” Cytokine. 55: 435-40, 2011). Moreover, experiments have shown that galectins, particularly galectin-1 (gal-1) and galectin-3 (gal-3), are directly involved in the pathogenesis of these classes of disease (Toussaint et al., “Galectin-1, a gene preferentially expressed at the tumor margin, promotes glioblastoma cell invasion.”, Mol. Cancer. 11:32, 2012, Liu et al 2012, Newlaczyl et al., “Galectin-3—a jack-of-all-trades in cancer,” Cancer Lett. 313: 123-128, 2011, Banh et al., “Tumor galectin-1 mediates tumor growth and metastasis through regulation of T-cell apoptosis,” Cancer Res. 71: 4423-31, 2011, Lefranc et al., “Galectin-1 mediated biochemical controls of melanoma and glioma aggressive behavior,” World J. Biol. Chem. 2: 193-201, 2011, Forsman et al., “Galectin 3 aggravates joint inflammation and destruction in antigen-induced arthritis,” Arthritis Reum. 63: 445-454, 2011, de Boer et al., “Galectin-3 in cardiac remodeling and heart failure,” Curr. Heart Fail. Rep. 7, 1-8, 2010, Ueland et al., “Galectin-3 in heart failure: high levels are associated with all-cause mortality,” Int J. Cardiol. 150: 361-364, 2011, Ohshima et al., “Galectin 3 and its binding protein in rheumatoid arthritis,” Arthritis Rheum. 48: 2788-2795, 2003).

High levels of serum Galectin-3 have been shown to be associated with some human diseases, such progressive heart failure, which makes identification of high-risk patients using galectin-3 testing an important part of patient care. Galectin-3 testing may be useful in helping physicians determine which patients are at higher risk of hospitalization or death. For example, the BGM Galectin-3® Test is an in vitro diagnostic device that quantitatively measures galectin-3 in serum or plasma and can be used in conjunction with clinical evaluation as an aid in assessing the prognosis of patients diagnosed with chronic heart failure. Measure of the concentration of endogenous protein galectin-3 can be used to predict or monitor disease progression or therapeutic efficacy in patients treated with cardiac resynchronization therapy (see U.S. Pat. No. 8,672,857, which is incorporated herein by reference in its entirety). Additionally, elevated galectin-3 levels have been associated with chronic renal failure, pulmonary hypertension, and cardiac arrhythmias.

Galectin-8 (gal-8) has been shown to be over-expressed in lung carcinomas and is in the invasive regions of xenografted glioblastomas.

Galectin-9 (gal-9) is believed to be involved in the control of lesions arising from immunoinflammatory diseases, and be generally implicated in inflammation. Gal-9 appears to mediate apoptosis in certain activated cells.

Aspects of the invention relate to compounds that bind galectins involved in human disorders, such as inflammatory diseases, fibrotic diseases, neoplastic diseases or combinations thereof. In some embodiments, the compounds bind galectins, including, but not limited to, galectin-1 (gal-1), galectin-3 (gal-3), galectin-8 (gal-8) and/or galectin-9 (gal-9).

### Galectin Inhibitors

Natural oligosaccharide ligands capable of binding to galectin-1 and/or galectin-3, for example, modified forms of pectins and galactomannan derived from Guar-gum have been described (see WO 2013040316, US 20110294755, WO 2015138438). Synthetic digalactosides like lactose, N-acetyllactosamine (LacNAc) and thiolactose effective against pulmonary fibrosis and other fibrotic disease (WO 2014067986 A1, incorporated herein by reference in their entireties).

Advances in protein crystallography and availability of high definition 3D structure of the carbohydrate recognition domain (CRD) of many galectins have generated many derivatives with enhanced affinity to the CRD having a greater affinity than galactose or lactose (WO 2014067986, incorporated herein by reference in its entirety). These compounds were shown to be effective for treatment of an animal model of lung fibrosis which is thought to mimic human idiopathic pulmonary fibrosis (IPF). For example a thio-digalactopyranosyl substituted with 3-fiuorophenyl-2,3-triazol groups (TD-139) has been reported to bind to galectin 3 and to be effective in in a mouse model of lung fibrosis. The compound required pulmonary administration using intra-tracheal instillation or nebulizers (see U.S. Pat Nos. 8,703,720, 7,700,763, 7,638,623 and 7,230,096, incorporated herein by reference in their entireties).

Aspects of the invention relates to novel compounds that mimic the natural ligand of galectin proteins. In some embodiments, the compound mimics the natural ligand of galectin-3. In some embodiments, the compound mimics the natural ligand of galectin-1. In some embodiments, the compound mimics the natural ligand of galectin-8. In some embodiments, the compound mimics the natural ligand of galectin-9.

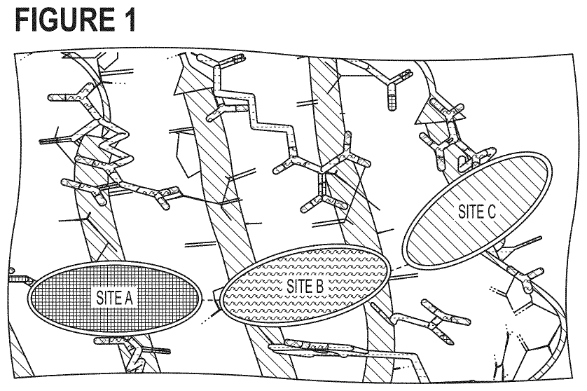

In some embodiments, the compound has a mono, di or oligomer structure composed of Galactose-Se core bound to the anomeric carbon on the galactose and which serves as a linker to the rest of the molecule. In some embodiments, the Galactose-Se core may be bound to other saccharide/amino acid/acids/group that bind galectin CRD (as shown in FIG. 1 in the high definition 3D structure of galectin-3) and together can enhance the compound's affinity to the CRD. In some embodiments, the Galactose-Se core may be bound to other saccharide/amino acid/acids/group that bind in “site B” of the galectin CRD (as shown in FIG. 1 in the high definition 3D structure of galectin-3) and together can enhance the compound's affinity to the CRD.

According to some aspects, the compounds can have substitutions that interact with site A and/or site C to further improve the association with the CRD and enhance their potential as a therapeutic targeted to galectin-dependent pathology. In some embodiments, the substituents can be selected through in-silico analysis (computer assisted molecular modeling) as described herein. In some embodiments, the substituents can be further screened using binding assay with the galectin protein of interest. For example, the compounds can be screened using a galectin-3 binding assay and/or an in-vitro inflammatory and fibrotic model of activated cultured macrophages (see Chávez-Galán, L. et al., Immunol. 2015; 6: 263).

According to some aspects, the compounds comprise one or more specific substitutions of the core Galactose-Se. For example, the core Galactose-Se can be substituted with specific substituents that interact with residues located within the CRD. Such substituents can dramatically increase the association and potential potency of the compound as well as the ‘drugability’ characteristic.

### Selenium

Selenium has five possible oxidation states (−2, 0, +2, +4 and +6), and therefore is well represented in a variety of compounds with diverse chemical properties. Furthermore, selenium can be present in the place of sulphur in virtually all sulphur compounds, inorganic as well as organic.

Most selenium compounds, organic and inorganic, are readily absorbed from the diet and transported to the liver—the prime organ for selenium metabolism. The general metabolism of selenium compounds follows three major routes depending on the chemical properties, that is, redox-active selenium compounds, precursors of methylselenol and seleno-amino acids.

Selenium is generally known as an antioxidant due to its presence in selenoproteins as selenocysteine, but can also toxic. The toxic effects of selenium are, however, strictly concentration and chemical species dependent. One class of selenium compounds is a potent inhibitor of cell growth with remarkable tumor specificity (Misra, 2015). Sodium Selenite has been studied as a cytotoxic agent in Advanced Carcinoma (SECAR, see Brodin, Ola et al., 2015).

### Galactoside-Selenium Compounds

Aspects of the invention relates to compounds comprising pyranosyl and/or furanosyl structures bound to a selenium atom on the anomeric carbon of the pyranosyl and/or furanosyl.

In some embodiments, specific aromatic substitutions can be added to the galactose core or heteroglycoside core to further enhance the affinity of the selenium bound pyranosyl and/or furanosyl structures. Such aromatic substitutions can enhance the interaction of the compound with amino acid residues (e.g. Arginine, Tryptophan, Histidine, Glutamic acid etc . . . ) composing the carbohydrate-recognition-domains (CRD) of the lectins and thus strengthen the association and binding specificity.

In some embodiments, the compound comprises monosaccharides, disaccharides and oligosaccharides of galactose or a heteroglycoside core bound to a selenium atom on the anomeric carbon of the galactose or of the heteroglycoside.

In some embodiments, the compound is a symmetric digalactoside wherein the two galactosides are bound by one or more selenium bonds. In some embodiments, the compound is a symmetric digalactoside wherein the two galactosides are bound by one or more selenium bonds and wherein the selenium is bound to the anomeric carbon of the galactose. In some embodiments, the compound is a symmetric digalactoside wherein the two galactosides are bound by one or more selenium bonds and one or more sulfur bonds and wherein the selenium is bound to the anomeric carbon of the galactose. Yet in other embodiments, the compound can be an asymmetric digalactoside. For example, the compound can have different aromatic or aliphatic substitutions on the galactose core.

In some embodiments, the compound is a symmetric galactoside wherein a single galactoside having one or more selenium on the anomeric carbon of the galactose. In some embodiments, the galactoside has one or more selenium bound to the anomeric carbon of the galactose and one or more sulfur bound to the selenium. In some embodiments, the compound can have different aromatic or aliphatic substitutions on the galactose core.

Without being bound to the theory, it is believed that the compounds containing the Se containing molecules render the compound metabolically stable while maintaining the chemical, physical and allosteric characteristics for specific interaction with lectins or galectins known to recognize carbohydrates. In some embodiments, the digalactoside or oligosaccharides of galactose of the present invention are metabolically more stable than compounds having an O-glycosidic bond.

In some embodiments, the digalactoside or oligosaccharides of galactose of the present invention are metabolically more stable than compounds having an S-glycosidic bond.

Aspects of the invention relate to compounds based on galactoside structure with a Selenium bridge [X] to another galactose, hydroxyl cyclohexane, aromatic moiety, alkyl, aryl, amine, or amide.

As used herein, the term “alkyl group” is meant to comprise from 1 to 12 carbon atoms, for example 1 to 7 or 1 to 4 carbon atoms. In some embodiments, the alkyl group may be straight- or branched-chain. In some embodiments, the alkyl group may also form a cycle comprising from 3 to 7 carbon atoms, preferably 3, 4, 5, 6, or 7 carbon atoms. Thus alkyl encompasses any of methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

As used herein, the term “alkenyl group” is meant to comprise from 2 to 12, for example 2 to 7 carbon atoms. The alkenyl group comprises at least one double bond. In some embodiments, the alkenyl group encompasses any any of vinyl, allyl, but-1-enyl, but-2-enyl, 2,2-dimethylethenyl, 2,2-dimethylprop-1-enyl, pent-1-enyl, pent-2-enyl, 2,3-dimethylbut-1-enyl, hex-1-enyl, hex-2-enyl, hex-3-enyl, prop-1,2-dienyl, 4-methylhex-1-enyl, cycloprop-1-enyl group, and others.

As used herein, the term “alkoxy group” relates to an alkoxy group containing 1-12 carbon atoms, which may include one or more unsaturated carbon atoms. In some embodiments the alkoxy group contains 1 to 7 or 1 to 4 carbon atoms, which may include one or more unsaturated carbon atoms. Thus the term “alkoxy group” encompasses a methoxy group, an ethoxy group, a propoxy group, a isopropoxy group, a n-butoxy group, a sec-butoxy group, tert-butoxy group, pentoxy group, isopentoxy group, 3-methylbutoxy group, 2,2-dimethylpropoxy group, n-hexoxy group, 2-methylpentoxy group, 2,2-dimethylbutoxy group 2,3-dimethylbutoxy group, n-heptoxy group, 2-methylhexoxy group, 2,2-dimethylpentoxy group, 2,3-dimethylpentoxy group, cyclopropoxy group, cyclobutoxy group, cyclopentyloxy group, cyclohexyloxy group, cycloheptyloxy group, and 1-methylcyclopropyloxy group.

As used herein, the term “aryl group” is meant to comprise from 4 to 12 carbon atoms. Said aryl group may be a phenyl group or a naphthyl group. The above-mentioned groups may naturally be substituted with any other known substituents within the art of organic chemistry. The groups may also be substituted with two or more of the said substituents. Examples of substituents are halogen, alkyl, alkenyl, alkoxy, nitro, sulfo, amino, hydroxy, and carbonyl groups. Halogen substituents can be bromo, fluoro, iodo, and chloro. Alkyl groups are as defined above containing 1 to 7 carbon atoms. Alkenyl are as defined above containing 2 to 7 carbon atoms, preferably 2 to 4. Alkoxy is as defined below containing 1 to 7 carbon atoms, preferably 1 to 4 carbon atoms, which may contain an unsaturated carbon atom. Combinations of substituents can be present such as trifluoromethyl.

As used herein, the term “heteroaryl group” is meant to comprise any aryl group comprising from 4 to 18 carbon atoms, wherein at least one atom of the ring is a heteroatom, i.e. not a carbon. In some embodiments, the heteroaryl group may be a pyridine, or an indole group.

The above-mentioned groups may be substituted with any other known substituents within the art of organic chemistry. The groups may also be substituted with two or more of the substituents. Examples of substituents are halogen, alkoxy, nitro, sulfo, amino, hydroxy, and carbonyl groups. Halogen substituents can be bromo, fluoro, iodo, and chloro. Alkyl groups are as defined above containing 1 to 7 carbon atoms. Alkenyl are as defined above containing 2 to 7 carbon atoms, for example 2 to 4. Alkoxy is as defined below containing 1 to 7 carbon atoms, for example 1 to 4 carbon atoms, which may contain an unsaturated carbon atom.

### Monomeric-Selenium Polyhydroxylated-Cycloalkanes

In some embodiments, the compound is a monomeric-selenium polyhydroxylated-cycloalkanes compound having Formula (1) or Formula (2) or a pharmaceutically acceptable salt or solvate thereof:

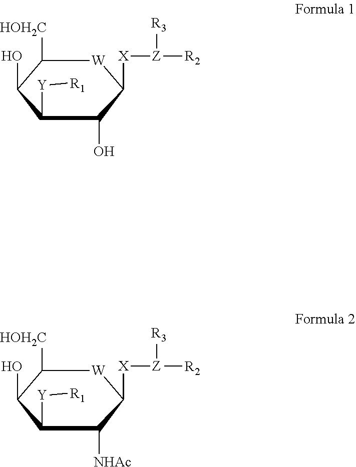

### Wherein X is Selenium;

Wherein Z is i selected from a carbohydrate or linkage consisting of O, S, C, NH, CH2, Se, amino acid to R₂ and R₃;
Wherein W is selected from the group consisting of O, N, S, CH2, NH, and Se;
Wherein Y is selected from the group consisting of O, S, C, NH, CH2, Se, amino acid and any combinations of the foregoing.
Wherein R₁, R₂, and R₃ are independently selected from the group consisting of CO, SO2, SO, PO2, PO, CH, Hydrogen, Hydrophobic linear and cyclic hydrocarbons including Heterocyclic substitutions of molecular weight of 50-200 D, including, but not limited to:
a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens;. Halogens can be a fluoro, a chloro, a bromo or an iodo group.
b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group;
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group; and
e) a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives; an amino group, a substituted amino group, an imino group, or a substituted imino group.

### Dimeric Selenium Polyhydroxylated-Cycloaklanes Compounds

In some embodiments, the compound is a dimeric-polyhydroxylated-cycloalkane compound.

In some embodiments, the compound has the general formulas (3) and (4) below or a pharmaceutically acceptable salt or solvate thereof:

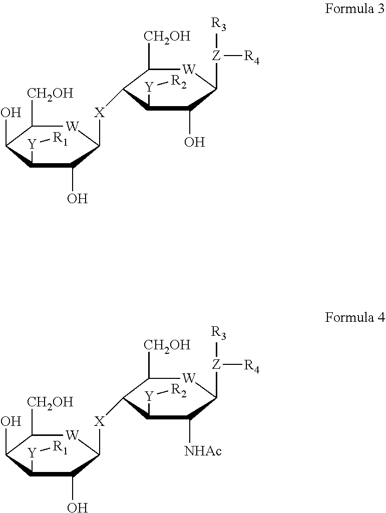

### Wherein X is Se, Se—Se or Se—S;

Wherein W is selected from the group consisting of O, N, S, CH2, NH, and Se;
Y and Z are selected from the group consisting of O, S, C, NH, CH2, Se and amino acid;
Wherein R₁, R₂, R₃, and R₄ are independently selected from the group consisting of CO, SO2, SO, PO2, PO, CH, Hydrogen, Hydrophobic linear and cyclic including Heterocyclic substitutions of molecular weight of about 50-200 D including, but not limited to:
a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted With an amino group, an alkyl group of at least 4 carbons substituted with both an amino and a carboxy group, an alkenyl group of at least 4 carbons substituted with both an amino and a carboxy group, and an alkyl group substituted with one or more halogens;
b) a phenyl group substituted with at least one car boxy group, a phenyl group substituted With at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group;
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group; and
e) saccharide; a substituted saccharide; D-galactose; substituted D-galactose; C3-[1,2,3]-triaZol-1-yl-substituted D-galactose; hydrogen, an alkyl group, an alkenyl group, an aryl group, a heteroaryl group, and a heterocycle and derivatives; an amino group, a substituted amino group, an imino group, or a substituted imino group.

### Oligomeric Selenium Polyhydroxylated-Cycloaklanes Compounds with 3 or More Units

In some embodiment, the compound is an oligomeric selenium polyhydroxylated-cycloalkane compound with 3 or more units. In some embodiments, the compound can have the general formulas (6) and (7) below or a pharmaceutically acceptable salt or solvate thereof

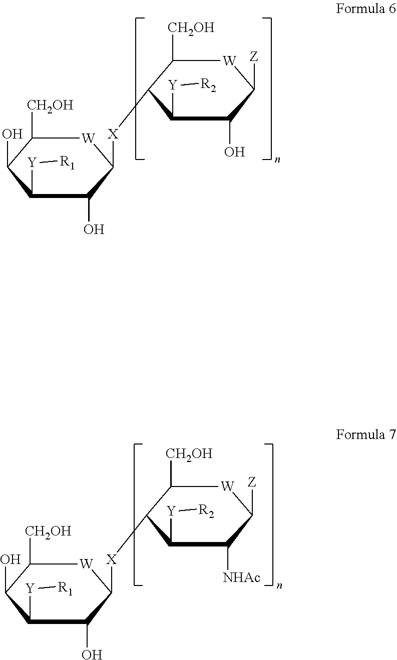

Wherein n≤24;

### Wherein X is Se, Se—Se or Se—S;

Wherein W is selected from the group consisting of O, N, S, CH2, NH, and Se;
Wherein Y and Z are independently selected from the group consisting of O, S, C, NH, CH2, Se, Amino acid;
Wherein R₁ and R₂ are independently selected from the group consisting of CO, SO2, SO, PO2, PO, CH, Hydrogen, Hydrophobic linear and cyclic including Heterocyclic substitutions of molecular weight of about 50-200 D including, but not limited to:
a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens;
b) a phenyl group substituted with at least one car boxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group;
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group;
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted With at least one substituted carbonyl group; and
e) a saccharide; a substituted saccharide; D-galactose; substituted D-galactose; C3-[1,2,3]-triaZol-1-yl-substituted D-galactose; hydrogen, an alkyl group, an alkenyl group, an aryl group, a heteroaryl group, and a heterocycle and derivatives; an amino group, a substituted amino group, an imino group, or a substituted imino group.

As used herein, the term “alkyl group” relates to an alkyl group containing 1-7 carbon atoms, which may include one or more unsaturated carbon atoms. In some embodiments the alkyl group contains 1-4 carbon atoms, which may include one or more unsaturated carbon atoms. The carbon atoms in the alkyl group may form a straight or branched chain. The carbon atoms in said alkyl group may also form a cycle containing 3, 4, 5, 6, or 7 carbon atoms. Thus, the term “alkyl group” used herein encompasses methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

In some embodiments, the compound has the following formulas and is an inhibitor of galectin-3: Table 1 shows non-limiting examples of monomeric Se Galactosides.

In some embodiments, the compound has the following formulas and is an inhibitor of galectin-3. Non-Limiting examples of mono-Se saccharides are shown in Table 1.

TABLE 1

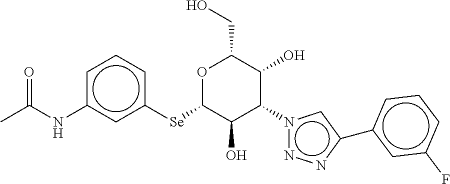

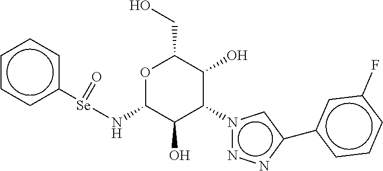

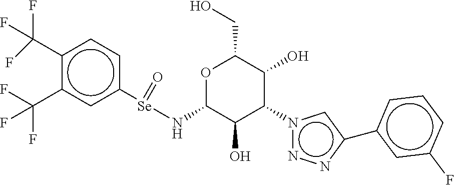

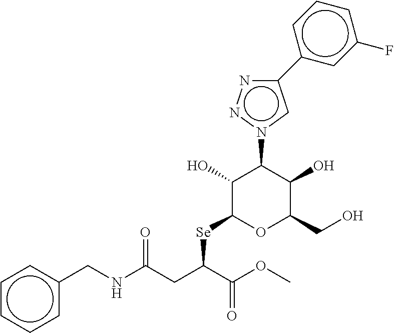

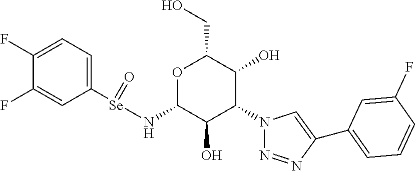 [SMILES: O=[Se](N[C@@H]1O[C@H](CO)[C@H](O)[C@H](n2cc(-c3cccc(F)c3)nn2)[C@H]1O)c1ccc(F)c(F)c1]

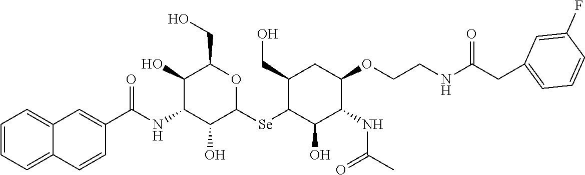 [SMILES: CC(=O)N[C@H]1[C@H](OCCNC(=O)Cc2cccc(F)c2)C[C@H](CO)C([Se]C2O[C@H](CO)[C@H](O)[C@@H](NC(=O)c3ccc4ccccc4c3)[C@H]2O)[C@@H]1O]

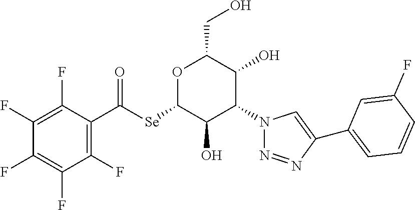 [SMILES: O=C([Se][C@@H]1O[C@H](CO)[C@H](O)[C@H](n2cc(-c3cccc(F)c3)nn2)[C@H]1O)c1c(F)c(F)c(F)c(F)c1F]

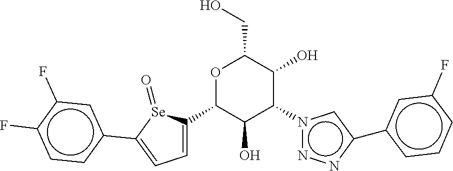

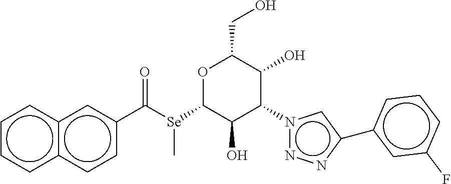

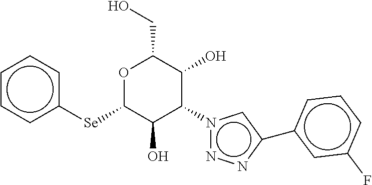

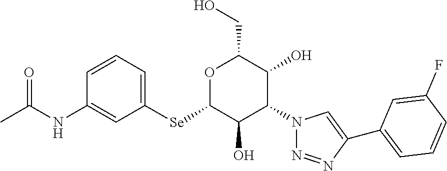 [SMILES: CC(=O)Nc1cccc([Se][C@@H]2O[C@H](CO)[C@H](O)[C@H](n3cc(-c4cccc(F)c4)nn3)[C@H]2O)c1]

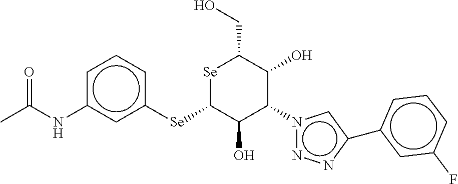

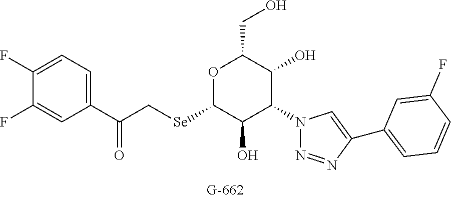 [SMILES: O=C(C[Se][C@@H]1O[C@H](CO)[C@H](O)[C@H](n2cc(-c3cccc(F)c3)nn2)[C@H]1O)c1ccc(F)c(F)c1]

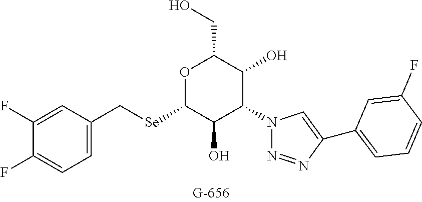 [SMILES: OC[C@H]1O[C@@H]([Se]Cc2ccc(F)c(F)c2)[C@H](O)[C@@H](n2cc(-c3cccc(F)c3)nn2)[C@H]1O]

In some embodiments, the compound has the following formulas and is an inhibitor of galectin-3. Table 2 shows non-limiting examples of Di—Se saccharides.

TABLE 2
Selenium di-saccharides

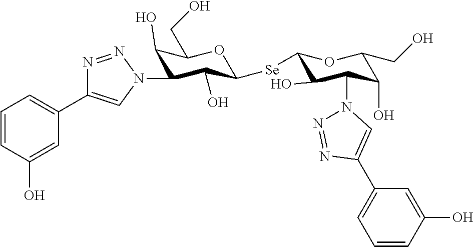

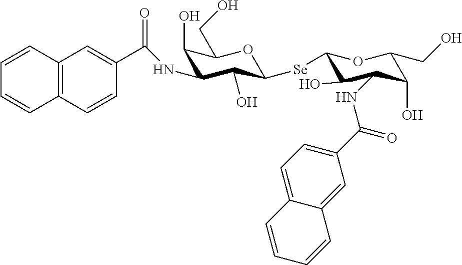

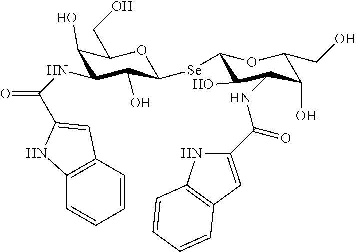

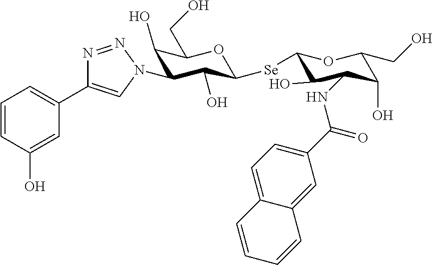

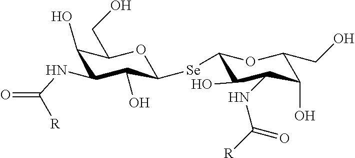

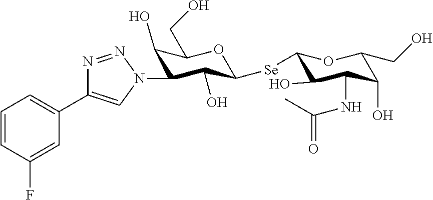

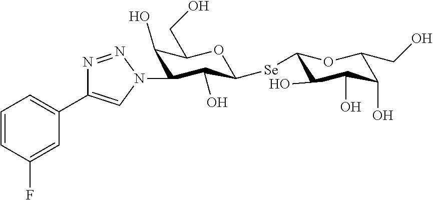

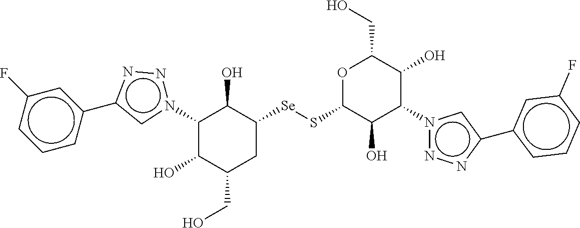

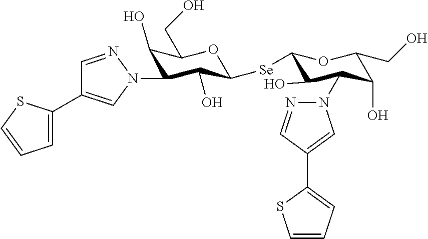

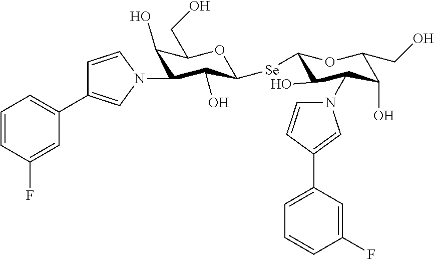

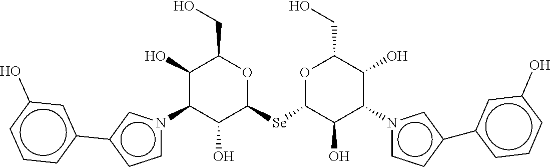

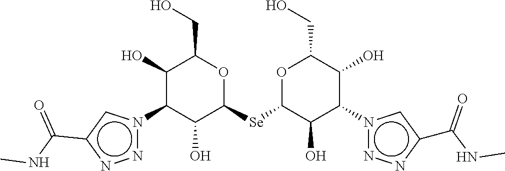

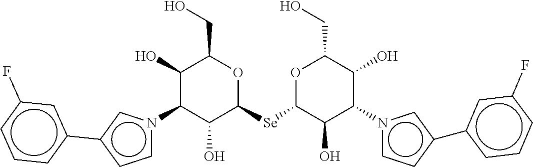

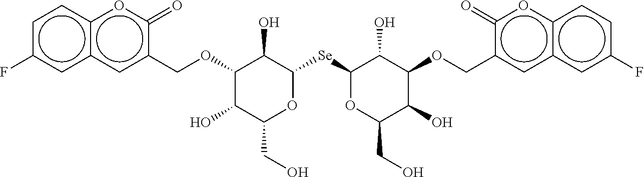

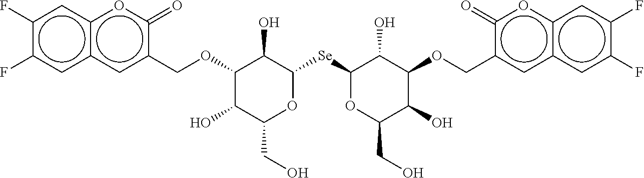

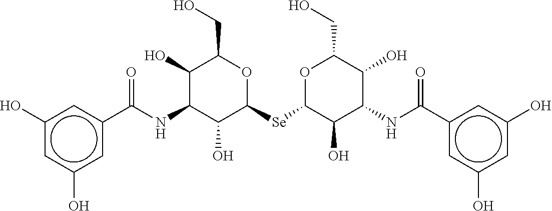

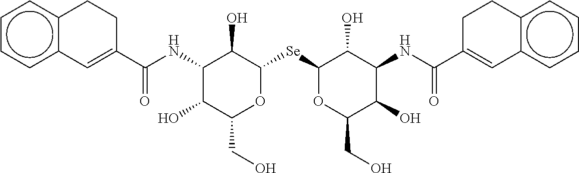

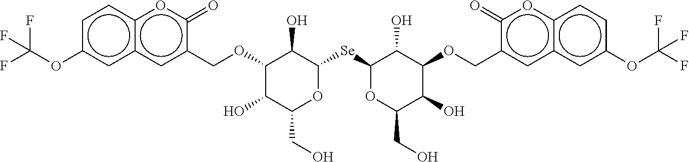

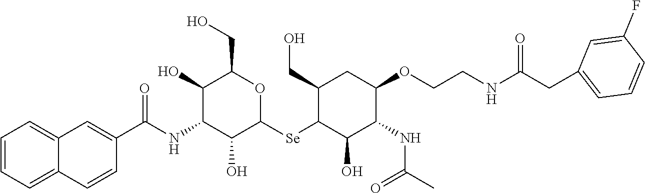 [SMILES: CC(=O)N[C@H]1[C@H](OCCNC(=O)Cc2cccc(F)c2)C[C@H](CO)C([Se]C2O[C@H](CO)[C@H](O)[C@@H](NC(=O)c3ccc4ccccc4c3)[C@H]2O)[C@@H]1O]

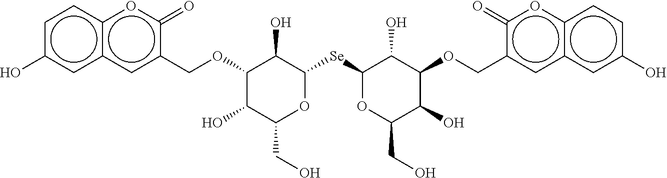

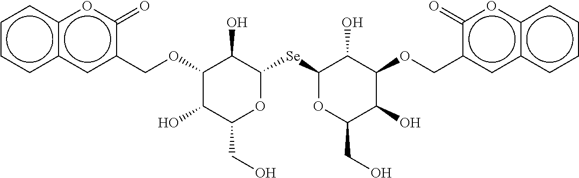

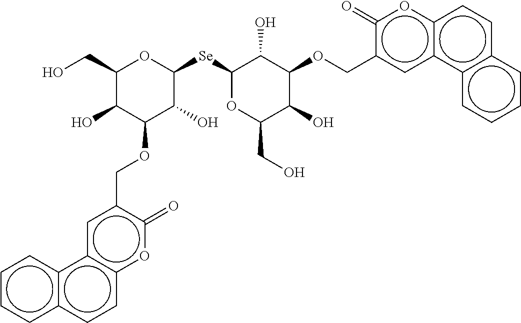

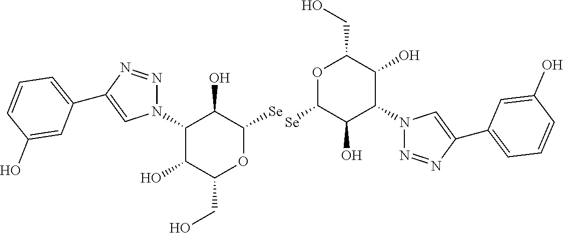

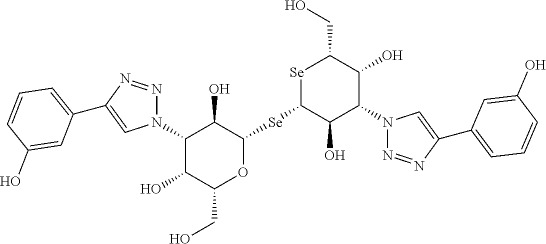 [SMILES: OC[C@H]1O[C@@H]([Se][C@@H]2[Se][C@H](CO)[C@H](O)[C@H](n3cc(-c4cccc(O)c4)nn3)[C@H]2O)[C@H](O)[C@@H](n2cc(-c3cccc(O)c3)nn2)[C@H]1O]

In some embodiments, the compound has the following formulas and is an inhibitor of galectin-3: Table 3 shows non-limiting examples of oligo-Se saccharides.

TABLE 3

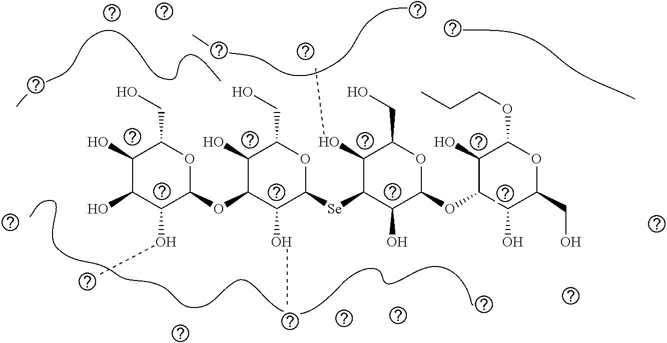

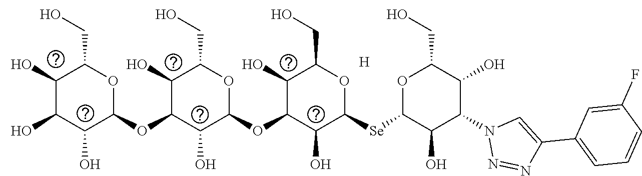

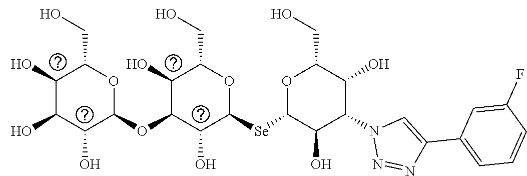 [SMILES: OC[C@@H]1O[C@@H](O[C@H]2[C@@H](O)[C@H](CO)O[C@@H]([Se][C@@H]3O[C@H](CO)[C@H](O)[C@H](n4cc(-c5cccc(F)c5)nn4)[C@H]3O)[C@@H]2O)[C@H](O)[C@@H](O)[C@H]1O]

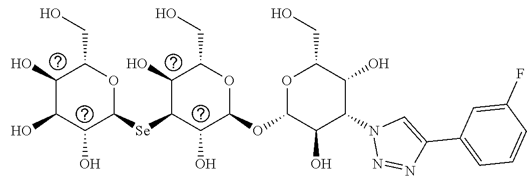 [SMILES: OC[C@@H]1O[C@@H]([Se][C@H]2[C@@H](O)[C@H](CO)O[C@@H](O[C@@H]3O[C@H](CO)[C@H](O)[C@H](n4cc(-c5cccc(F)c5)nn4)[C@H]3O)[C@@H]2O)[C@H](O)[C@@H](O)[C@H]1O]

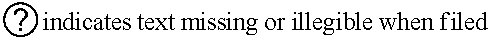

Tetrameric Se-galactosides are expected to have higher affinity to the CRD versus the trimeric structure due to additional potential interaction of hydroxyl groups with amino-acids in the CRD vicinity (see Example 14).

Without being bound to the theory, the galactose-selenium compounds described herein have an enhanced stability as its structure is less prone to hydrolysis (metabolism) and oxidation, e.g. aromatic ring without substitutions, Carbon-Oxygen systems, Carbone-Nitrogen system etc;

### Computational Scoring of Ligand-Protein Affinity

Standard assays to evaluate the binding ability of the ligand toward target molecules are known in the art, including for example, ELISAs, western blots and RIAs. Suitable assays are described in detail herein. In some embodiments, the binding kinetics (e.g., binding affinity) can be assessed by standard assays known in the art such as by Biacore analysis. Assays to evaluate the effects of the compounds on functional properties of the galectin are described in further detail herein.

One way to determine protein-ligand binding affinity uses a structure-based model that can predict the interaction of the protein-ligand complex that results when the ligand binds to the protein. Such structures may be studied by x-ray crystallography. In some embodiments, compounds of interest can be screened “in silico” to predict the ligand's affinity to the lectin or galectin proteins using any scoring system known in the art.

In some embodiments, a computational modeling can be used to facilitate structure-based drug design. The in-silico model also enables to visually inspect the protein-compound interaction, conformational strain and possible steno clashes and avoid them. In some embodiments, the protein-ligand affinity can be scored using a Glide (Schrödinger, Portland Oreg.). The combination of position and orientation of a ligand relative to the protein, along with the flexible docking, is referred to as a ligand pose and scoring of the ligand pose for Glide is done with GlideScore. GlideScore is a quantitative measurement that provides an estimate for a ligand binding free energy. It has many terms, including force field (electrostatic, van der Waals, etc . . . ) contributions and terms rewarding or penalizing interactions known to influence ligand binding. It contains two energetic elements; the enthalpic and entropic contributions of a biological reaction. The thermodynamic rationale for enthalpy-entropy compensation is based on the fact that, as the binding becomes stronger, enthalpy becomes more negative and entropy concomitantly tends to decrease due the formation of a tight complex. As such, ligands having the lowest GlideScore can be selected.

The methods and compounds are provided for the inhibition of Galectin-3 and/or Galectin-1, however the in-silico model, assays and compounds described herein may be applied to other galectin proteins and lectins.

An in-silico model of Galectin-3 CRD based on the 1 KJR crystal structure of human Galectin-3 CRD (Sorme, P. et al. (2005) J.Am.Chem.Soc. 127: 1737-1743) and improved using Galectin-3 known “actives” and “inactive” compounds as a training and test sets was used. The 1KJR crystal structure was selected due to its unique extended cavity that allows for larger substitutes (e.g. indole or naphtalen) on the C3 position of the galactose (Vargas-Berebgurl 2013, Barondes 1998, Sorme 2003). Table 4 shows the GlideScore for the different di-galactosides: (1) thiogalactoside, galactoside, selenogalactoside, diselenogalactoside having identical substituents.

TABLE 4

| | | ELISA of | | |
|---|---|---|---|---|
| | | Galectin-3 | | |
| | Com- | binding | FP | |
| | pound | Inhibition | assay | Glide- |
| Compound | name | (μg/ml) | (μg/ml) | Score |
| 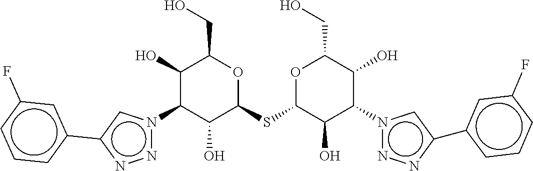 | TD-139 Galecto Biotech | 0.03 | 0.3 | −6.289 |
| 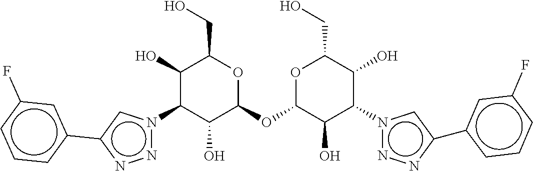 | | | | −5.675 |
| 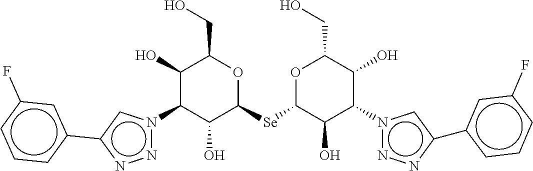 | G-625 | 0.01 | 0.18 | −6.254 |
| 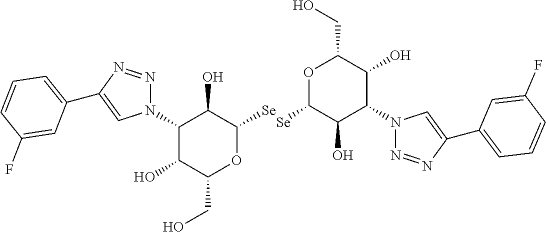 | G-626 | 0.9 | 4.1 | −5.494 |

The GlideScore data showed that the introduction of Se to the anomeric carbon (G-625) on the galactose scores the same as the thiogalactoside (TD-139, also referred as G-240). The results also showed that the thiogalactoside (TD-139) and the selenogalactoside compound (G-625) have comparable overall estimated predictor of free energy. As such, the thiogalactoside (TD-139) and the selenogalactoside compound (G-625) are expected to have comparable affinity to galectin-3 and inhibitor effects.

These compounds were tested for their affinity with integrins and with galectin-3. Surprisingly, the selenogalactoside compound (G-625) showed from about at least 2 to about at least 3 times better affinity to galectin-3 and to integrins.

The Se atom allows the rest of the molecule (for example G-625) to fulfill the interactions seen with TD-139, but with a superior affinity to Galectin-3 vs. TD-139 as was shown in the Elisa based assay and fluorescent polarization assay. In some embodiments, the selenogalactoside of formula (1) has an affinity to galectin-3 that is at least twice or at least three time stronger than TD-139. In some embodiments, the selenogalactosides of the present invention have an affinity to galectin-3 that is at least twice or at least three times stronger than the corresponding thiogalactoside.

The ‘drugability’ characteristic, as defined by the computational structure analysis considers compound's: (1) stereoisomerization, (2) position of the hydroxyl groups on the sugar (e.g. axial or equatorial) and (3) position and nature of substituents.

1) Stereoisomerization: It should be noted that compounds with identical 2D nomenclature can have a different 3D structure that can lead to a very different binding pose as well as different predicting binding free energy predictor, GlideScore.

2) Hydroxyl groups: The position of the hydroxyl groups on the sugar (e.g. axial or equatorial) play an important role in compounds binding. Specifically, the present invention relates to compounds that are galactose-based bound to a Selenium atom bound to the anomeric carbon, serving as a linker to the rest of the molecule.

3) Substituents: According to some aspects, the compounds can have substituents capable of, or designed to, reach amino acids that are part of the binding site which were known and unknown to play a role in ligand's binding. One of skill in the art would appreciate that galectins bind the monosaccharide galactose with dissociation constants in the millimolar range. It has been shown that addition of N-acetyl glucosamine to galactose can provide additional interaction with neighboring sites boosts the compound affinity to galectin-3 over 10 fold (Bachhawat-Sikder Et al. FEBS Lett. 2001 Jun 29;500(1-2):75-9).

Further addition of non-natural derivatives, such as naphtol, at the 3 position of saccharides, can enhance the affinity to the low micromolar range, e.g. 0.003 mM. This substitution exploits cation-π interactions with the surface residue Arg 144.

Human Galectin-3 cavity is shallow with high solvent accessibility. It is very hydrophilic but capable of forming cation-π interactions with Arg144 and possibly Trp181 (Magnani 2009, Logan 2011). It has been shown that upon ligand's binding, Arg144 moves 3.5 A upwards from the protein surface to make a pocket for the Arene-Arginine interaction. It should be noted that Arg144 is absent in other galectin, e.g. Gal-1, Gal-9 and this is being exploited in our in-silico model. Similarly, potency can be improved by exploiting cation-π interactions with the surface residue of Arg186. For example, triazole substitution at C3 of galactose has been reported to increase Galectin 3 affinity (Salameh B A et al. Bioorg. Med. Chem. Lett. 2005 Jul 15; 15(14):3344-6.)

Tryptophan 181 at subsite C is conserved throughout the galectin family. A π-π stacking interaction between the Trp181 (W181) side chain and a carbohydrate residue (galactose being the natural carbohydrate occupant) accommodated within subsite C occurs in all reported galectin-saccharide complexes.

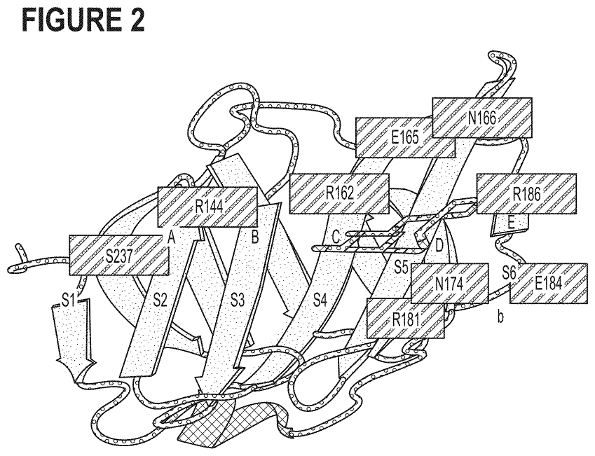

To develop effective approaches for the structure-based design of potent galectin inhibitors, such as galectin-3 inhibitors, it is important to understand the detailed molecular basis for carbohydrate recognition, based on the three dimensional structure and physiochemical properties of the conserved binding motif. High-resolution structural information greatly aids in this respect (see Ultra-High-Resolution Structures and Water Dynamics, Saraboji, K. et al., Biochemistry. 2012 Jan 10; 51(1): 296-306.). While it is clear that the galectin-3 CRD site is pre-organized to recognize a carbohydrate like framework of oxygens (see FIG. 2), it was not expected to recognize Se containing compounds with a two-fold to a three-fold increased activity.

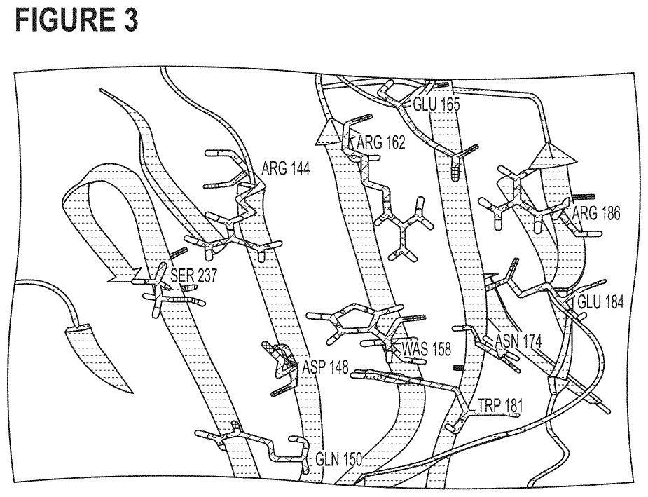

In Galectin-3 (See CRD binding pocket in FIG. 3), the side chain of Arg144 is capable of adopting different conformations due to its inherent flexibility that could contribute to greater affinity via an arginine-arene interaction (a cation-π or π-π stacking interaction) with the aromatic moiety.

In some embodiments, galectin's key residues that affect ligand affinity were identified using computational alanine scanning mutagenesis (ASM) or an “in-silico-alanine-scan”. ASM can be performed by sequential replacement of individual residues by alanine to identify residues involved in protein function, stability and shape. Each alanine substitution examines the contribution of an individual amino add to the functionality of the protein.

To better understand the importance of residues within the CRD binding pocket (FIG. 3) an “in-silico-alanine-scan” was run by docking in Glide the compound of Formula 1 and a galectin-3 inhibitor, 3,3′-Dideoxy-3,3′-di-[4-(3-fluorophenyl)-lH-l,2,3-triazol-l-yl]-l,l′-sulfanediyl-di-D-galactopyranoside (TD139, see WO2016005311A1, incorporated by reference in its entirety). Residues that were predicted to be involved in the binding were mutated and it was expected that the mutations to alanine would have an effect on the GlideScore results. The Alanine Scan was used to predict the importance of residues to the ligand's binding.

For example, it was reported that Galectin-3 R186S abolishes carbohydrate interactions. The R186S was shown to have has a selectively lost affinity for LacNAc, a disaccharide moiety commonly found on glycoprotein glycans, and has lost the ability to activate neutrophil leukocytes and intracellular targeting into vesicles. (see Salomonsson E. et al., J Biol Chem. 2010 Nov 5;285(45):35079-91.)

TABLE 5
In-silico Alanine scan comparison results using TD-139 Compound

| Compound | Substitution | GlideScore | dG |
|---|---|---|---|
| TD-139 | Galectin-3 WT | −6.289 | 100.00 |
| TD-139 | Galectin-3-R186A | −5.345 | 84.99 |
| TD-139 | Galectin-3-R162A | −5.56 | 88.41 |
| TD-139 | Galectin-3-R144A | −6.502 | 103.39 |
| TD-139 | Galectin-3-W181A | −5.256 | 83.57 |
| TD-139 | Galectin-3-H158A | −5.315 | 84.51 |
| TD-139 | Galectin-3-N174A | −5.069 | 80.60 |

TABLE 6
In-silico Alanine scan comparison results
using G-625 Compound having Formula 1

| Compound | Substitution | GlideScore | dG |
|---|---|---|---|
| G-625 | Galectin-3 WT | −6.254 | 100 |
| G-625 | Galectin-3-R186A | −5.989 | 95.76 |
| G-625 | Galectin-3-R162A | −5.637 | 90.13 |
| G-625 | Galectin-3-R144A | −6.564 | 104.96 |
| G-625 | Galectin-3-W181A | −5.37 | 85.87 |
| G-625 | Galectin-3-H158A | −5.178 | 82.80 |
| G-625 | Galectin-3-N174A | −5.074 | 81.13 |

** dG > 100 suggests increase in ligand binding upon mutation to Alanine while dG < 100 suggests decrease in ligand binding upon mutation.

These results suggest that the ‘molecular interaction profile’ of TD-139 differs from that of G-625. Tables 5 and 6 show the interaction profile as predicted by the in-silico model. TD139 is greatly affected by the introduction of R186A mutation (there is “˜15% reduction” in the GlideScore which is a predictor for the free binding energy). On the other hand R186A has less of an effect on G-625 and G-625 is more sensitive to H158A mutation.

Surprisingly, the Alanine scan showed that residue N174 play an important role in the binding of both TD-139 and G-625 compounds. Without being bound to the theory it is possible that residue N174 may help in positioning the Galactose core in ‘the optimal orientation’ that will enable the CRD site to recognize carbohydrate like framework of the oxygens.

The in-silco Alanine scan suggested that G-625 has a unique binding profile while maintaining the interactions with known CRD residues like Arg 162, Arg 186 and Arg 144. Based on these results the interactions with residues located at Site A: S237; Site B: D148; Site C-D: A146, K176, G182 and E165; and N166 in Site C-loop (FIGS. 2 and 3) were explored to improve the interaction with the CRD.

### Synthetic Route

The compounds of this invention may be prepared by the following general methods and procedures. It should be appreciated that where typical or preferred process conditions (e.g. reaction temperatures, times, molar ratios of reactants, solvents, pressures, pH etc) are given, other process conditions may also be used unless otherwise stated. Optimum reaction conditions may vary with the particular reactants, solvents used and pH etc., but such conditions can be determined by one skilled in the art by routine optimization procedures.

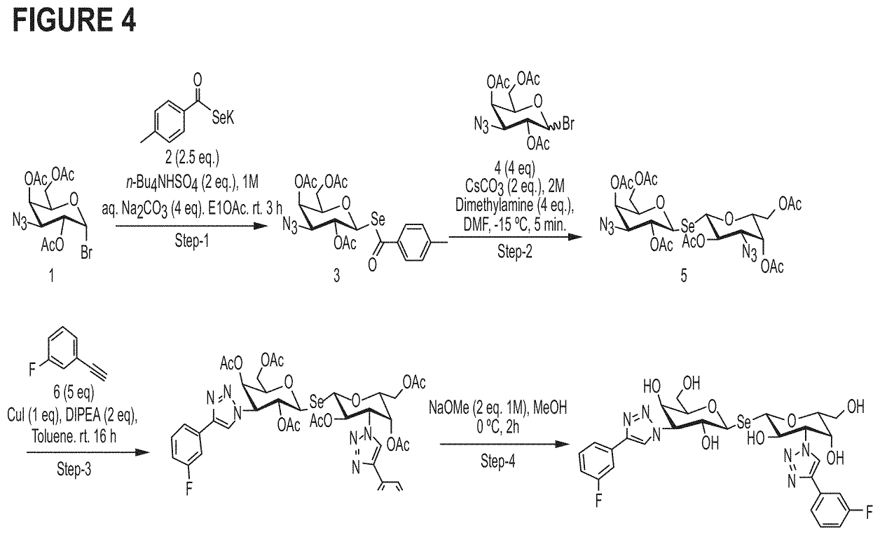

In some embodiments, the compound was synthetized using the synthetic route shown in FIG. 4.

For example, compound G-625 was prepared as shown in Example 17.

### Pharmaceutical Compositions

Aspects of the invention relate to the use of the compounds described herein for the manufacture of medicaments.

Aspects of the invention relate to pharmaceutical compositions comprising one or more of the compounds described herein. In some embodiments, the pharmaceutical compositions comprise one or more of the following: pharmaceutically acceptable adjuvant, diluent, excipient, and carrier.

The term “pharmaceutically acceptable carrier” refers to a carrier or adjuvant that may be administered to a subject (e.g., a patient), together with a compound of this invention, and which does not destroy the pharmacological activity thereof and is nontoxic when administered in doses sufficient to deliver a therapeutic amount or an effective mount of the compound.

“Pharmaceutically acceptable carrier” refers to any and all solvents, dispersion media. The use of such media and compounds for pharmaceutically active substances is well known in the art. Preferably, the carrier is suitable for oral, intravenous, intramuscular, subcutaneous, parenteral, spinal or epidural administration (e.g., by injection or infusion). Depending on the route of administration, the active compound can be coated in a material to protect the compound from the action of acids and other natural conditions that can inactivate the compound.

In some embodiments, the pharmaceutical composition comprises a compound described herein as active ingredient together with a pharmaceutically acceptable adjuvant, diluent, excipient or carrier. A pharmaceutical composition can comprise from 1 to 99 weight % of a pharmaceutically acceptable adjuvant, diluent, excipient or carrier and from 1 to 99 weight % of a compound described herein.

The adjuvants, diluents, excipients and/or carriers that may be used in the composition of the invention are pharmaceutically acceptable, i.e. are compatible with the compounds and the other ingredients of the pharmaceutical composition, and not deleterious to the recipient thereof. The adjuvants, diluents, excipients and carriers that may be used in the pharmaceutical composition of the invention are well known to a person within the art.

An effective oral dose of the compound of the present invention to an experimental animal or human may be formulated with a variety of excipients and additives that enhance the absorption of the compound via the stomach and small intestine.

The pharmaceutical composition of the present invention may comprise two or more compounds of the present invention. The composition may also be used together with other medicaments within the art for the treatment of related disorders.

In some embodiments, the pharmaceutical composition comprising one or more compounds described herein may be adapted for oral, intravenous, topical, intraperitoneal, nasal, buccal, sublingual, or subcutaneous administration, or for administration via the respiratory tract in the form of, for example, an aerosol or an air-suspended fine powder, or, for administration via the eye, intra-ocularly, intravitreally or corneally.

In some embodiments, the pharmaceutical composition comprising one or more compounds described herein may be in the form of, for example, tablets, capsules, powders, solutions for injection, solutions for spraying, ointments, transdermal patches or suppositories.

Some aspects of the present invention relate to pharmaceutical composition comprising the compound described herein or a pharmaceutically acceptable salt or solvate thereof and optionally a pharmaceutically acceptable additive, such as carrier or excipient.

An effective oral dose could be 10 times and up to 100 times the amount of the effective parental dose.

An effective oral dose may be given daily, in one or divided doses or twice, three times weekly, or monthly.

In some embodiments, the compounds described herein can be co-administered with one or more other therapeutic agents. In certain embodiments, the additional agents may be administered separately, as part of a multiple dose regimen, from the compounds of this invention (e.g., sequentially, e.g., on different overlapping schedules with the administration of the compound of the invention. In other embodiments, these agents may be part of a single dosage form, mixed together with the compounds of this invention in a single composition. In still another embodiment, these agents can be given as a separate dose that is administered at about the same time that the compound of the invention. When the compositions include a combination of the compound of this invention and one or more additional therapeutic or prophylactic agents, both the compound and the additional agent can be present at dosage levels of between about 1 to 100%, and more preferably between about 5 to 95% of the dosage normally administered in a monotherapy regimen.

Aspects of the invention relates to a composition or a compound to treat neoplastic conditions in combination with other anti-neoplastic drugs including but not limited to checkpoint inhibitors (anti-CTLA2, anti-PD1, anti-PDL1), other immune modifiers including but not limited to anti-OX40, and multiple other anti-neoplastic agents of multiple mechanisms.

In some embodiments, a therapeutically effective amount of the compound or of the composition can be compatible and effective in combination with a therapeutically effective amount of various anti-inflammatory drugs, vitamins, other pharmaceuticals and nutraceuticals drugs or supplement, or combinations thereof without limitation.

Aspects of the invention relates to a composition or a compound to treat neoplastic conditions in combination with other anti-neoplastic drugs including but not limited to checkpoint inhibitors (anti-CTLA2, anti-PD1, anti-PDL1), other immune modifiers including but not limited to anti-OX40, and multiple other anti-neoplastic agents of multiple mechanisms.

### Methods of Treatment

Some aspects of the invention relate to the use of the compounds described herein or the composition described herein for use in the treatment of a disorder relating to the binding of a galectin to a ligand. In some embodiments, galectin is galectin-3.

Some aspects of the invention relate to the method of treating various disorders relating to the binding of a galectin to a ligand. In some embodiments, the methods comprise administering in a subject in need thereof a therapeutically effective amount of at least one compound described herein. In some embodiments, the subject in need thereof is a human having high levels of galectin-3. Levels of galectin, for example galectin-3 can be quantified using any methods known in the art.

In some embodiments, the disorder is an inflammatory disorder, for example inflammatory bowel disease, Crohn's disease, multiple sclerosis, systemic lupus erythematosus, or ulcerative colitis.

In some embodiments, the disorder is fibrosis, for example liver fibrosis, pulmonary fibrosis, kidney fibrosis, heart fibrosis or fibrosis of any organ compromising the normal function of the organ.

In some embodiments, the disorder is cancer.

In some embodiments, the disorder is an autoimmune disease such as rheumatoid arthritis and multiple sclerosis.

In some embodiments, the disorder is heart disease or heart failure.

In some embodiments, the disorder is a metabolic disorder, for example diabetes.

In some embodiments, the disorder relating is pathological angiogenesis, such as ocular angiogenesis, disease or conditions associated with ocular angiogenesis and cancer.

In some embodiments, the composition or the compound can be used in the treatment of nonalcoholic steatohepatitis with or without liver fibrosis, inflammatory and autoimmune disorders, neoplastic conditions or cancers.

In some embodiments, the composition can be used in the treatment of liver fibrosis, kidney fibrosis, lung fibrosis, or heart fibrosis.

In some embodiments, the composition or the compound is capable of enhancing anti-fibrosis activity in organs, including but not limited to, liver, kidney, lung, and heart.

In some embodiments, the composition or the compound can be used in treatment of inflammatory disorders of the vasculature including atherosclerosis and pulmonary hypertension.

In some embodiments, the composition or the compound can be used in the treatment of heart disorders including heart failure, arrhythmias, and uremic cardiomyopathy.

In some embodiments, the composition or the compound can be used in the treatment of kidney diseases including glomerulopathies and interstitial nephritis.

In some embodiments, the composition or the compound can be used in the treatment of inflammatory, proliferative and fibrotic skin disorders including but not limited to psoriasis and scleroderma.

Aspects of the invention relates to methods of treating allergic or atopic conditions, including but not limited to eczema, atopic dermatitis, or asthma.

Aspects of the invention relates to methods of treating inflammatory and fibrotic disorders in which galectins are at least in part involved in the pathogenesis, by enhancing anti-fibrosis activity in organs, including but not limited to liver, kidney, lung, and heart.

Aspects of the invention relates to methods relates to a composition or a compound that has a therapeutic activity to treat nonalcoholic steatohepatitis (NASH). In other aspects, the invention elates to a method to reduce the pathology and disease activity associated with nonalcoholic steatohepatitis (NASH).

Aspects of the invention relates to a composition or a compound used in treating or a method of treating inflammatory and autoimmune disorders in which galectins are at least in part involved in the pathogenesis including but not limited to arthritis, systemic lupus erythematosus, rheumatoid arthritis, asthma, and inflammatory bowel disease.

Aspects of the invention relates to a composition or a compound to treat neoplastic conditions (e.g. benign or malignant neoplastic diseases) in which galectins are at least in part involved in the pathogenesis by inhibiting processes promoted by the increase in galectins. In some embodiments, the invention relates a method of treating neoplastic conditions (e.g. benign or malignant neoplastic diseases) in which galectins are at least in part involved in the pathogenesis by inhibiting processes promoted by the increase in galectins. In some embodiments, the composition or a compound can be used to treat or prevent tumor cell growth, invasion, metastasis, and neovascularization. In some embodiments, the composition or a compound can be used to treat primary and secondary cancers.

### EXAMPLES

### Example 1: Compound Inhibition of Galectin Binding to Physiologic Ligands

Galectin proteins, including but not limited to galectin-3 and galectin-1, have multiple biologically relevant binding ligands in mammalian species, including but not limited to rodents, primates, and humans. Galectins are carbohydrate-binding proteins that bind to glycoproteins with 3-galactoside-containing sugars. The result of binding of galectin proteins to these ligands results in a plethora of biological effects in and on cells and in tissues and whole organisms including regulating cell survival and signaling, influencing cell growth and chemotaxis, interfering with cytokine secretion, mediating cell-cell and cell-matrix interactions or influencing tumor progression and metastasis. Additionally, changes in normal expression of galectin proteins are responsible for pathological effects in multiple diseases, including but not limited to inflammatory, fibrotic and neoplastic diseases.

Compounds described in this invention are designed to bind to the carbohydrate recognition domain of galectin proteins, including but not limited to galectin-3, and disrupt interactions with biologically relevant ligands. They are intended to inhibit the function of galectin proteins that may be involved in pathological processes at normal levels of expression or in situations where they are increased over physiological levels.

Some of the ligands for galectin proteins that are important in normal cellular function and pathology in disease include, but are not limited to, TIM-3 (T cell immunoglobulin mucin-3), CD8, T cell receptor, integrins, galectin-3 binding protein, TGF-β receptor, laminins, fibronectins, BCR (B cell receptor, CTLA-4 (cytotoxic T-lymphocyte-associated protein-4), EGFR (Epidermal growth factor receptor), FGFR (fibroblast growth factor receptor), GLUT-2 (glucose transporter-2), IGFR (insulin-like growth factor receptor), various interleukins, LPG (lipophosphoglycan), MHC (major histocompatibility complex), PDGFR (platelet-derived growth factor receptor), TCR (T cell receptor), TGF-β (transforming growth factor-β), TGFβR (transforming growth factor-β receptor, CD98, Mac3 antigen (Lysosome-associated membrane protein 2 (LAMP2) also known as CD107b (Cluster of Differentiation 107b)).

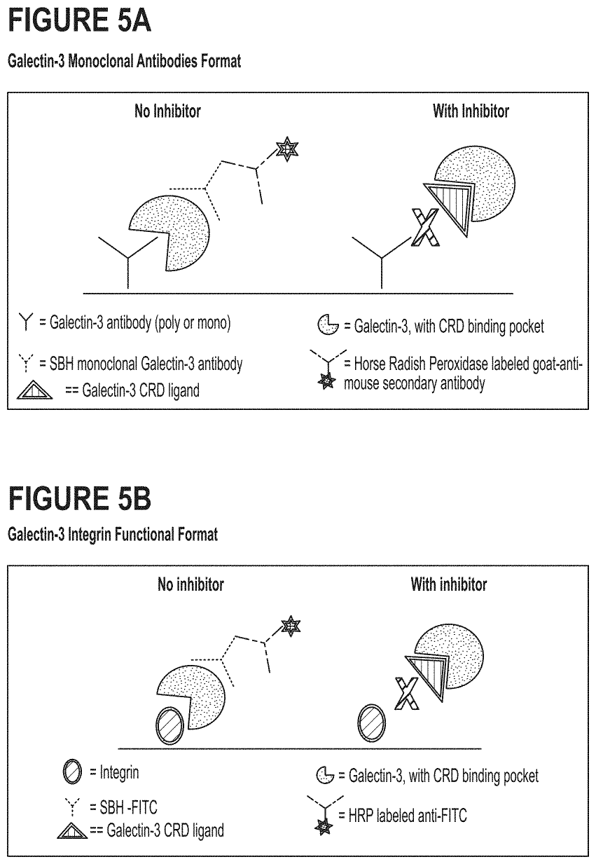

Experiments have been performed to evaluate the physical interaction of galectin proteins with these various biological ligands mediating cellular functions. The experiments were designed to evaluate the interaction between various galectin-3 ligands and determine whether compounds described herein are able to inhibit these interactions, as shown in FIGS. 5A and 5B.

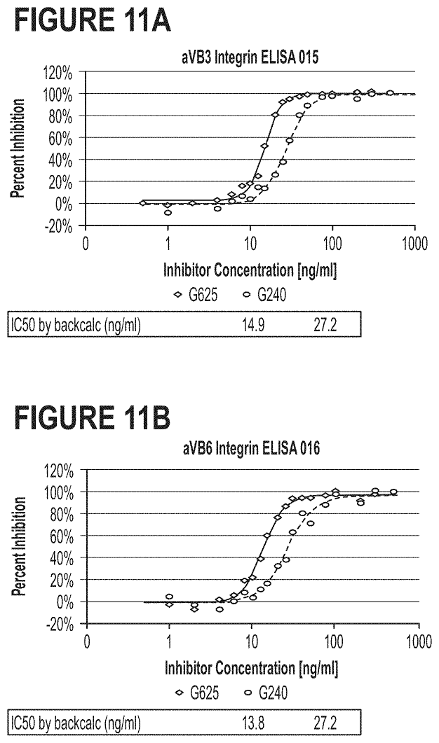

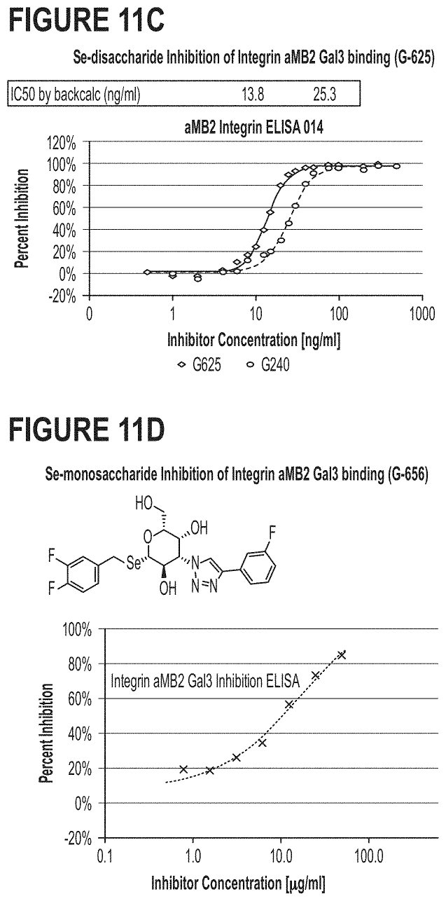

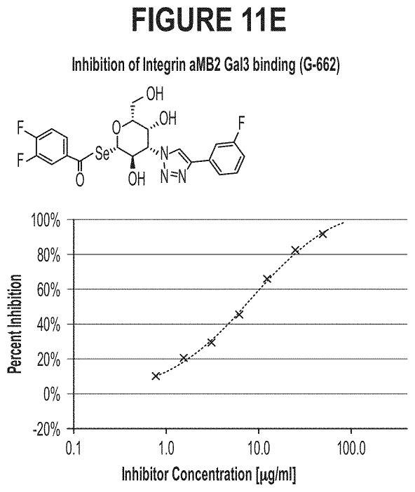

Using this assay, the compounds described herein were shown to inhibit the interaction of galectin proteins with their ligands, including but not limited to various integrin molecules (αVβ3, αVβ6, αMβ2, α2β3, and others) with IC50's in the range of about 0.5 nM to about 50 μM. In some embodiments, the IC50 is about from 0.5 nM to about 1 nM. In some embodiments, the IC50 is from about 1 nM to about 10 nM. In some embodiments, the IC50 is from about 10 nM to about 100 nM. In some embodiments, the IC50 is from about 100 nM to about 1 μM. In some embodiments, the IC50 is from about 1 μM to about 10 μM. In some embodiments, the IC50 is from about 10 μM to about 50 μM. See FIGS. 11A through 11E.

### Example 2: Compound Inhibition of Galectin Binding to Labeled Probes

Fluorescein-labeled probes have been developed which bind to galectin-3 and other galectin proteins and these probes have been used to establish assays that measure the binding affinity of ligands for the galectin proteins using Fluorescence Polarization (Sörme P, et al. Anal Biochem. 2004 Nov 1;334(1):36-47).

Compounds described herein avidly bind to galectin-3, as well as other galectin proteins, using this assay and displace the probe with high affinity, with IC₅₀'s (concentration at 50% inhibition) of between about 0.5 nM to about 5 μM. In some embodiments, the IC50 is about from 0.5 nM to about 1 nM. In some embodiments, the IC50 is from about 1 nM to about 10 nM. In some embodiments, the IC50 is from about 10 nM to about 100 nM. In some embodiments, the IC50 is from about 100 nM to about 1 μM. In some embodiments, the IC50 is from about 1 μM to about 10 μM. In some embodiments, the IC50 is from about 10 μM to about 20 μM.

### Inhibition of Physiologic Ligands

A functional assay was developed to test the inhibition of physiologic ligands such as integrins, as shown in FIG. 5B.

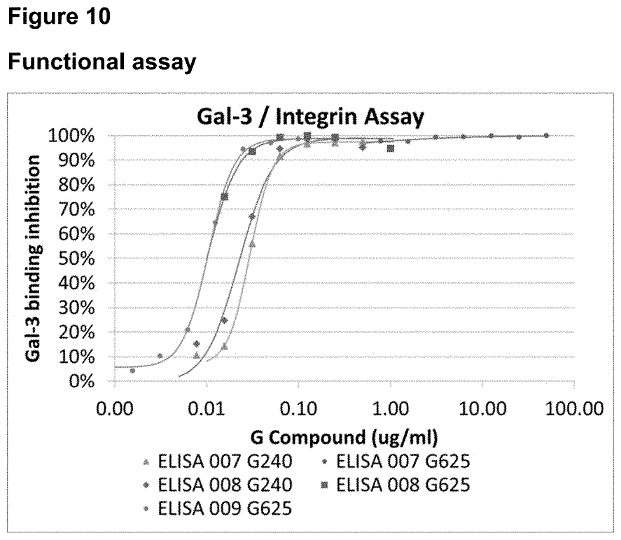

The thiodiglycoside G240 (TD-139) and the selanodiglycoside G-625 compound were compared using a gal-3/integrin interaction ELISA assay. FIG. 10 and FIGS. 11A-11C showed that G625 was more potent inhibitor of Gal-3/integrins than TD-139 (G240).

Se-monogalatosides (G-656 and G662) substituted with difluoride benzene have been shown to significantly inhibit the interaction of gal-3 with integrin as shown in FIG. 11D and 11E.

### Fluorescent Polarization

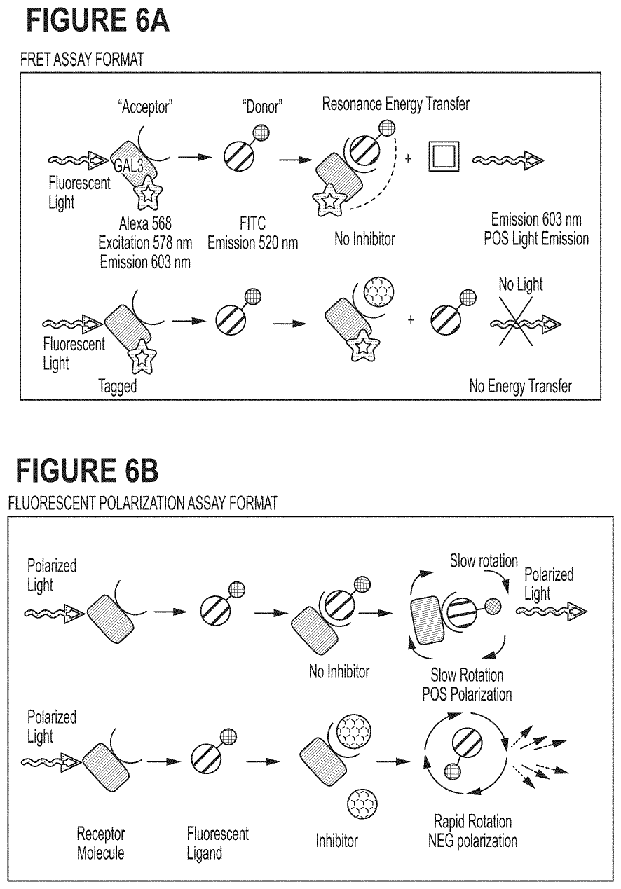

Two compounds (G-625 and G-240) were tested using a Fluorescent Polarization signal of specific Fluorescent ligand (See FIG. 6B).

### Structure

G-240 or TD-139: beta-D-Galactopyranoside, 3-deoxy-3-(4-(3-fluorophenyl)-1H-1,2,3-triazol-1-yl)-beta-D-galactopyranosyl 3-deoxy-3-(4-(3-fluorophenyl)-1H-1,2,3-triazol-1-yl)-1-thio-. G-240 (TD-139) has a sulfate bridge between two Aryl-triazol-galactosides.

G-625—beta-D-Galactopyranoside, 3-deoxy-3-(4-(3-fluorophenyl)-1H-1,2,3-triazol-1-yl)-beta-D-galactopyranosyl 3-deoxy-3-(4-(3-fluorophenyl)-1H-1,2,3-triazol-1-yl)-1-seleno-. G-625 has single selenide bridge between two Aryl-triazole-galactosides

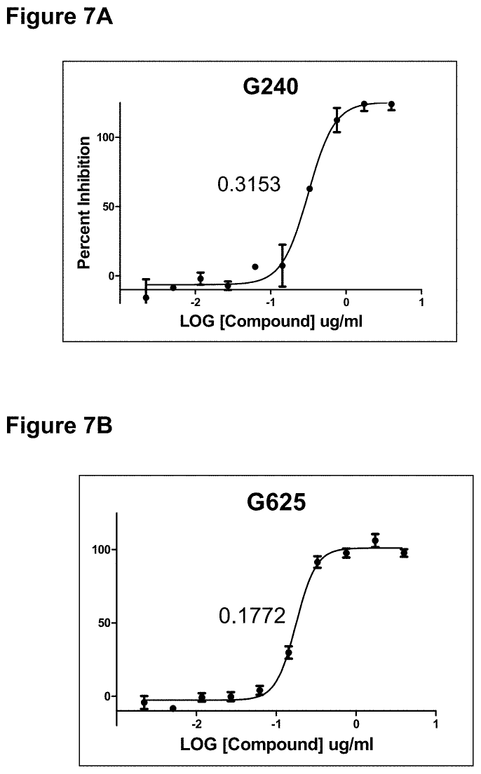

The inhibition curves showed in FIG. 7A and FIG. 7B showed that the compound described herein G-625 was twice better inhibitor then G-240 (TD-139) of Galectin-3 CRD specific fluorescent-ligand.

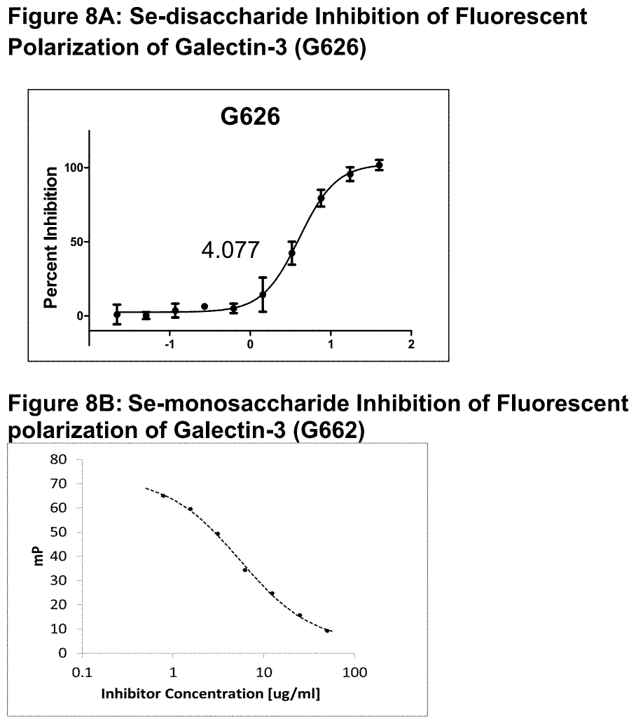

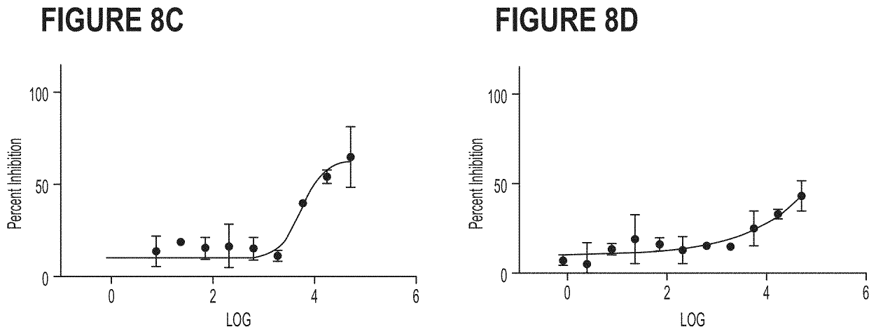

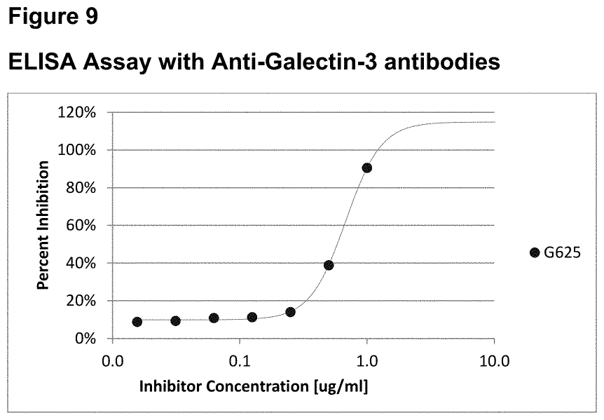

G-626, a diselenide derivative of G-625 was synthesized (see Table 4). G-626 showed an inhibitory activity in the Fluorescent polarization assay (see FIG. 6B and FIG. 8A).

G-662 a seleno-monosaccharide was synthesized (see Table 1) and shown to inhibit the Gal-3 binding in the Fluorescent Polarization assay FIG. 8B.

### Example 3: Compound Inhibition of Galectin Binding Using FRET Assay

FRET assay (fluorescent resonance energy transfer) assays were developed for evaluating the interaction of galectin proteins, including but not limited to galectin-3, with a model fluorescent-labeled probe (see FIG. 6A). Using this assay, compounds described herein avidly bind to galectin-3, as well as other galectin proteins, and displace the probe with high affinity, with IC₅₀'s (concentration at 50% inhibition) of between about 0.5 nM to about 5 μM. In some embodiments, the IC50 is about from 0.5 nM to about 1 nM. In some embodiments, the IC50 is from about 1 nM to about 10 nM. In some embodiments, the IC50 is from about 10 nM to about 100 nM. In some embodiments, the IC50 is from about 100 nM to about 1 μM. In some embodiments, the IC50 is from about 1 μM to about 5 μM.

### Example 4: Compound Binding to Amino Acid Residues in Galectin Proteins

Heteronuclear NMR spectroscopy is used to evaluate the interaction of compounds described herein with galectin molecules, including but not limited to galectin-3, to assess the interaction residues on the galectin-3 molecule.

Uniformly ¹⁵N-labeled Gal-3 is expressed in BL21 (DE3) competent cells (Novagen), grown in minimal media, purified over a lactose affinity column, and fractionated on a gel filtration column, as described previously for production of Gal-1 (Nesmelova I V, Pang M, Baum L G, Mayo K H. 1H, 13C, and 15N backbone and side-chain chemical shift assignments for the 29 kDa human galectin-1 protein dimer. Biomol NMR Assign 2008 Dec;2(2):203-205).

Uniformly ¹⁵N-labeled Gal-3 is dissolved at a concentration of 2 mg/ml in 20 mM potassium phosphate buffer at pH 7.0, made up using a 95% H₂O/5% D₂O mixture. ¹H-¹⁵N HSQC NMR experiments are used to investigate binding of a series of compounds described herein. ¹H and ¹⁵N resonance assignments for recombinant human Gal-3 were previously reported (Ippel H, et al. (1)H, (13)C, and (15)N backbone and side-chain chemical shift assignments for the 36 proline-containing, full length 29 kDa human chimera-type galectin-3. Biomol NMR Assign 2015 Apr;9(1):59-63.).

NMR experiments are carried out at 30° C. on Bruker 600 MHz, 700 MHz or 850 MHz spectrometers equipped with H/C/N triple-resonance probes and x/y/z triple-axis pulse field gradient units. A gradient sensitivity-enhanced version of two-dimensional ¹H-¹⁵N HSQC is applied with 256 (t1)×2048 (t2) complex data points in nitrogen and proton dimensions, respectively. Raw data are converted and processed by using NMRPipe and were analyzed by using NMRview.

These experiments show differences between compounds described herein in the binding residues in the carbohydrate binding domain of galectin-3.

### Example 5: Cellular Activity of Cytokine Activity Related to Galectin Binding Inhibition

Example 1 describes the ability of compounds of this application to inhibit the binding of physiologic ligands to galectin molecules. In the experiments of this example, the functional implications of those binding interactions were evaluated.

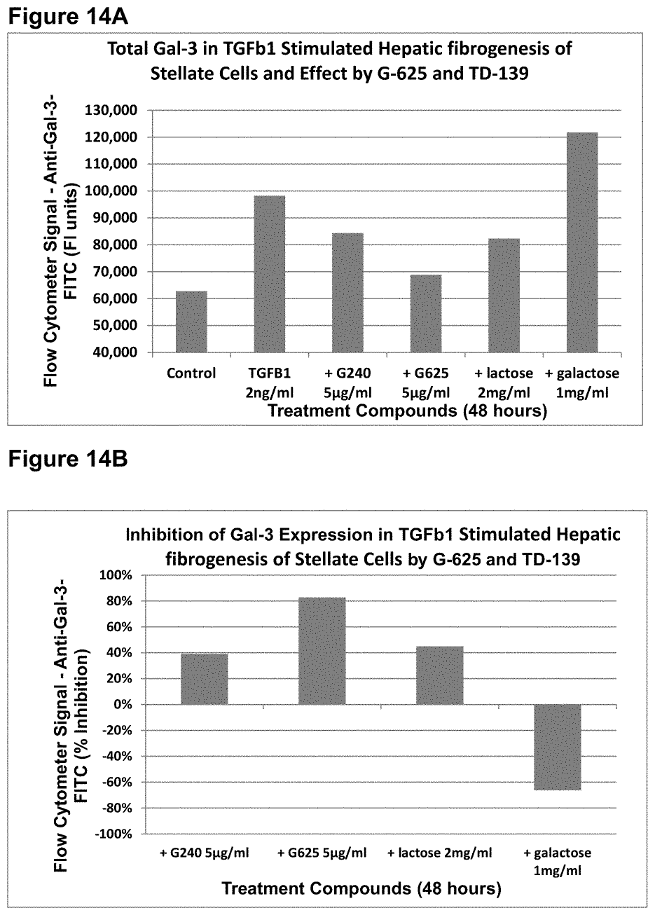

One of the interactions with galectin-3 that is inhibited by the compounds described herein was TGF-β receptor. Therefore, experiments were done to evaluate the effect of compounds on TGR-β receptor activity in cell lines. Various TGF-β responsive cell lines, including but not limited to LX-2 and THP-1 cells, were treated with TGF-β and response of the cells was measured by looking at activation of second messenger systems, including but not limited to phosphorylation of various intracellular SMAD proteins. After establishing that TGF-β activates the second messenger systems in the various cell lines, the cells were treated with compounds described herein. These experiments showed that these compounds inhibit TGF-β signaling pathways, confirming that the binding interaction inhibition described in Example 1 has a physiological role in cellular models. FIG. 14A and 14B show the enhanced activity of G-625 versus G-240.

Cellular assays were also performed to evaluate the physiological significance of inhibiting the interaction of galectin-3 with various integrin molecules. Cell-cell interaction studies were performed using monocytes binding to vascular endothelial cells, as well as other cell lines. Treatment of cells with compounds described herein was found to inhibit these integrin-dependent interactions, confirming that the binding interaction inhibition described in Example 1 had a physiological role in cellular models.

### Bioassay Procedures

Procedure for MCF-7 Cells (colon cancer) was as follow:

1. MCF-7 cells were resuspended in culture media containing 4× Pen/Strep and 0.25% Fetal Bovine Serum (Gibco lot# 1202161).
2. 100 ul media was added with approximately 4,000-10,000 cells/well, passage # 5 up to 30) and cells were incubated for at least 24 hrs at 37° C.
3. Tested compound was diluted serially in assay media as above, usually at a range of 100 μg/ml to 20 ng/mL
4. 100 ml serial diluted compound was added in duplicate to cells in assay plate. Final volume of each well was 200 ml, (containing 2× Pen/Strep, 0.25% FBS and compound as indicated
5. Cells were incubated 60-80 hours at 37° C.
6. 20 ml of Promega Substrate [CellTiter 96 Aqueous One Solution] Reagent was added to each well.
7. Cells were incubated 37° C. for 4-8 hrs and read OD at 490 nm.

Procedure for HTB-38 Cells (Breast cancer) was as follow:

1. HTB-38 cells were resuspended in culture media containing 8 ng/ml h-IFN-gamma, 4× Pen/Strep and 10% Fetal Bovine Serum (Gibco lot# 1260930).
2. Cells were transferred at 100μl/well in assay plate (4,000-10,000 cells/well, passage# 4-30).
3. Tested compound was diluted serially in assay media as above, usually in range of 100 μg/ml to 20 ng/mL
4. 100 μl/well serial diluted compound was added in duplicate to cells. Final volume of each well was 200 μl, containing 4 ng/ml h-IFN-gamma, 2× Pen/Strep,
5. Cells were Incubate 60-90 hours at 37° C.
6. 20 μl of Promega Substrate [CellTiter 96 Aqueous One Solution] Reagent was added to each well.
7. Cells were incubated at 37° C. for 4-8 hrs and read OD at 490 nm.

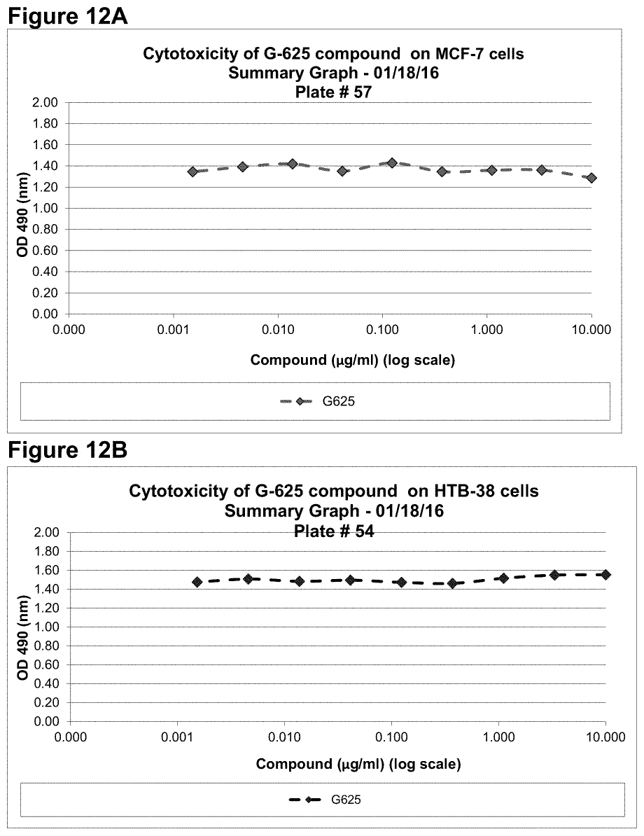

FIG. 12A and 12B viability of cell cultures in the present of the Se-digalactoside G-625 showed no cytotoxicity at concentration that have significant effect on inflammatory and fibrogenesis cell based models. Cells were exposed to the G-625 over 3 days in standard growth media.

Cellular motility assays are performed to evaluate the physiological significance of inhibiting the interaction of galectin-3 with various integrin and other cell surface molecules defined in Example 1. Cellular studies are performed using multiple cell lines in a semi-permeable membrane separated well apparatus. Treatment of cells with compounds described herein is found to inhibit cellular motility, confirming that the binding interaction inhibition described in Example 1 has a physiological role in cellular models.

### Example 6: In-Vitro Inflammatory Model (A Monocyte Based Assay)

A model of macrophage polarization was set up, starting from THP-1 monocytes culture which is differentiated into inflammatory macrophages using PMA (Phorbol 12-myristate 13-acetate) for 2-4 days. Once differentiated (MO macrophages), the macrophages were induced with LPS or LPS and IFN-gamma for macrophage activation (M1) to inflammatory stage for 1-3 days. Array of cytokines and chemokines were analyzed to confirm the polarization of THP-1-derived macrophages to inflammatory stage. The impact of the anti-galectin 3 compounds on macrophage polarization was assessed first by monitoring cell viability using a colorimetric method (using a tetrazolium reagent) to determine the number of viable cells in proliferation or cytotoxicity assays (Promega, The CellTiter 96® AQueous One Solution Cell Proliferation Assay which contains a novel tetrazolium compound [3-(4,5-dimethyl-2-yl)-5-(3-carboxymethoxyphenyl)-2-(4-sulfophenyl)-2H-tetrazolium, inner salt; MTS] and an electron coupling reagent (phenazine ethosulfate; PES)) and inflammatory stage evaluated by a quantitatively measure of the chemokine Monocyte Chemoattractant Protein-1 (MCP-1/CCL2), a key protein that regulates migration and infiltration of monocytes/macrophages in cellular process of inflammation. Follow-up testing for the expression and secretion of other cytokines and chemokines were done for leading active compounds. Results are expressed in percentage reduction of MCP-1.

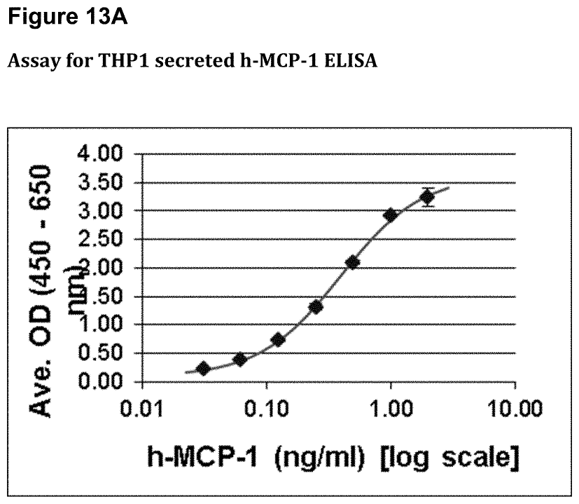

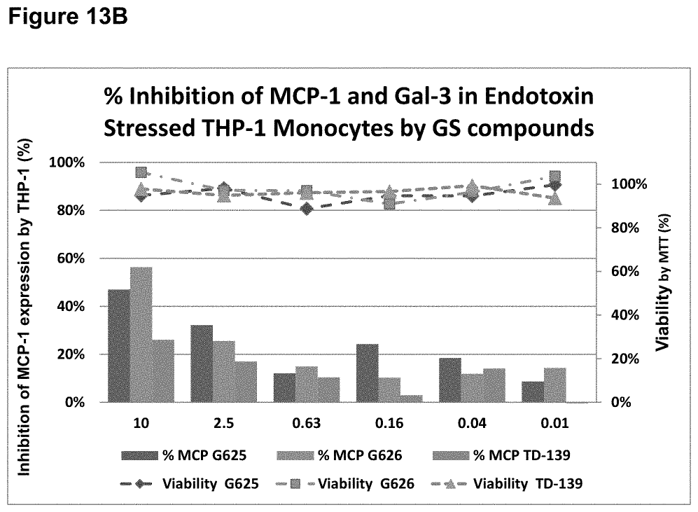

FIGS. 13A and 13B show inhibition of MCP-1 in inflammatory THP-1 monocytes stimulated with endotoxin for 5 days. THP-1 cells were stimulated by microbial endotoxin which transforms the cells to inflammatory macrophages (M1) which secret inflammatory cytokines like Monocyte Chemoattractant Protein-1 (MCP-1).

In this Example the method steps were as followed:

THP-1 cells were cultured in media containing Gentamicin
2) THP-1 cells are transfer to wells in a 96 well plate 2,000 cells/well for 2 days incubation in assay media containing 10 ng/ml PMA
3) Serial dilution of test compounds is made in LPS (10 ng/ml) containing media
4) To each well 100 ml of compounds/LPS solution is added to a final assay volume of each well of 200 ml which contains also Gentamicin and 5 ng/ml PMA
5) Cells are incubated up to 8 days.
6) Every other day samples of 60 ul are removed for bio-assay
7) At termination 15 ml of Promega Substrate CellTiter 96 Aqueous One Solution is added to each well to monitor cytotoxicity (at 490 nm)
8) For cellular biomarkers evaluation the cells are washed 1× PBS and extracted with 200 ul of Lysis buffer for 1 hour. Extract is spinned down 10 minutes and 120 ul sample is removed from top. All samples are kept at −70 C until testing.

FIG. 13 shows that both G-625 and G-626 have inhibitory effect on the inflammatory stage by reducing the secretion of MCP-1 a biomarker for polarized macrophage.

### Example 7: Cell Culture Fibrogenesis Model

Experiments were performed with fibrogenic stellate cell cultures, including but not limited to LX-2 cells, to evaluate the cellular effect of compounds herein. LX-2 cells were activated in culture using serum deprived media and media spiked with different percentages of THP-1 cell conditioned media. Activation of LX-2 cells was monitored by various well defined markers, including but not limited to TIMP-1. Demonstrable LX-2 cell activation was evident by 24 hours after treatment. The treatment of cells with compounds described herein was found to inhibit activation, confirming a physiological role in cellular models.

FIGS. 14A and 14B show inhibition of galectin-3 expression by the selenium compound G625 in TGFb1 in 5 days serum starved stimulated LX-2 cells, Hepatic fibrogenesis Stellate Cells.

TGFb1 stimulates hepatic stellate cells into the fibrogenesis pathway leading to secretion of collagen and other fibrosis biomarkers. Expression of galectin-3 on the hepatic cell membrane was greatly enhanced as the Flow Cytometer experiment has established using fluorescent tagged monoclonal antibodies to Gal-3. Lactose and Galactose were used to demonstrate the specificity of the stimulation to the expression of Gal-3. While it is known that lactose has binding affinity to Gal-3, galactose lacks this affinity. It was expected that lactose would have effect (at relatively high concentrations) while galactose should not have any effect. The result confirmed this hypothesis.

### Example 8: In Vivo Animal Models of Liver Fibrosis

### NASH Mouse Fibrosis Model

The NASH model uses male newborn mice [C57BL/6J mice]. The disease is induced by a single subcutaneous injection of streptozotocin (Sigma, St. Louis, Mo.) solution 2 days after birth which induced diabetes followed by administration of a high fat diet. Other models of NASH may also be used including the use of high fat and/or fat plus sugar diets in various strains of mice (DIAMOND mice). After four weeks of age a high fat diet (HFD, 57% of kcal from fat) is introduced for 12 and up to 16 weeks. Vehicle and test substances at the various doses are administered orally or SQ or intravenously weekly and calculated as mg/kg body weight.

Randomization of mice into treatment groups is done prior to treatment based on the plasma ALT levels and body weight. At minimum 3 treatment groups (of between 6 and 15 mice each) are in a study, including one group that is a vehicle control, one group that are normal mice, and the other groups contain various concentrations of seleno-galactoside compounds given at various intervals starting at various times during the development of NASH and liver fibrosis.

The seleno-galactoside compounds described herein, following various durations of treatments, reduce liver fibrosis as measured by collagen 10% to 80% versus the vehicle control or to almost normal collagen levels, liver fat levels by between 10% and 80%, liver cell apoptosis by between 10% and 80%, and liver inflammation by between 10% and 80%, as established in the normal mice.

### General Biochemical Tests

Liver functions are evaluated in Plasma for levels of AST, ALT, total bilirubin, creatinine, and TG are measured by example FUJI DRY CHEM 7000 (Fuji Film, Japan).

Liver biochemistry: To quantify liver hydroxyproline content, a quantitative assessment of collagen content, frozen liver samples (40-70 mg) are processed by a standard alkaline-acid hydrolysis method and hydroxyproline content is normalized to total liver proteins.

Total liver lipid-extracts are obtained from caudate lobes by Folch's method and liver TG levels are measured using the Triglyceride E-test (Wako, Japan).

Histopathological and immunohistochemical analyses liver sections are cut from paraffin blocks of liver tissue prefixed in Bouin's solution and stained with Lillie-Mayer's Hematoxylin (Muto Pure Chemicals, Japan) and eosin solution (Wako, Japan).

To visualize collagen deposition, Bouin's fixed liver sections are stained using picro-Sirius red solution (Waldeck GmbH & Co. KG, Germany). NAFLD Activity score (NAS) is also calculated according to established criteria.

Immunohistochemistry for SMA, F4/80, Galectin-3, CD36 and iNOS can be estimated from each positive area as indication for the extent of inflammation and fibrosis.

### Rat Fibrosis/Cirrhosis Model (TAA Model)

These experiments use male Sprague-Dawley rats between 160 and 280 g obtained from animal research facility (Jackson Laboratory) which are maintained according to the Guide for the Care and Use of Laboratory Animals (Institute of Laboratory Animal Resources, 1996, Nat. Acad. Press) and Institutional Animal Care and Use committee (IACUC). At the end of experiments, animals are euthanized under phenobarbital anesthesia.

After an acclimation period of two weeks, an eight week induction period is initiated, in which all rats are subjected to intraperitoneal (IP) injections Thioacetamide (TAA, Sigma Chemical Co., St. Louis, Mo., USA) of sterile solutions of dissolved in 0.9% saline, administered by IP injection twice or trice weekly with initial week dosage of 450 mg/kg/wk, followed by seven weeks regimen of 400 mg/kg/wk body weight. To assess for the progression of fibrosis two rats are euthanized at weeks 4 and 8, and the liver examined histologically. To develop cirrhosis animals are administered TAA intraperitoneally (IP) up to 11-12 weeks, for fibrosis 8 weeks are enough. Treatment is for 4 weeks beginning in week 8, vehicle control group is administered 0.9% NaCl intraperitoneally twice weekly for four weeks. Experimental test articles are given intraperitoneally, intravenously or orally twice or once a week, or at other intervals, beginning in week 8 or 11 for fibrosis or cirrhosis respectively. At the end of the treatment period, rats are placed under anesthesia using isofluorane between 1-5% through inhalation and a laparotomy is performed. At the time of sacrifice, portal pressure is measured using a 16 G angiocatheter introduced into the portal vein to measure the height of a water column. The liver is removed, weighed, and pieces from the largest lobes are used for further analysis. The spleen is also removed and weighed before being discarded.

Representative histology of Sirius red stained liver sections from experiment shows a 20% reduction in mean collagen which is statistical acceptable for anti-fibrosis effect. Strands of bridging fibrosis indicate advance fibrosis stage (these are strands of collagen fibers).

Biochemical Tests: As in the NASH model various diagnostic tests are done to evaluate the extend of liver damage due to the fibrosis:

Liver functions are evaluated in Plasma for levels of AST, ALT, total bilirubin, creatinine, and TG are measured by example FUJI DRY CHEM 7000 (Fuji Film, Japan).

Liver biochemistry : To quantify liver hydroxyproline content, a quantitative assessment of collagen content, frozen liver samples (40-70 mg) are processed by a standard alkaline-acid hydrolysis method and hydroxyproline content is normalized to total liver proteins.

Total liver lipid-extracts are obtained from caudate lobes by Folch's method and liver TG levels are measured using the Triglyceride E-test (Wako, Japan).

Histopathological and immunohistochemical analyses liver sections are cut from paraffin blocks of liver tissue prefixed in Bouin's solution and stained with Lillie-Mayer's Hematoxylin (Muto Pure Chemicals, Japan) and eosin solution (Wako, Japan).

To visualize collagen deposition, Bouin's fixed liver sections are stained using picro-Sirius red solution (Waldeck GmbH & Co. KG, Germany). NAFLD Activity score (NAS) is also calculated according to established criteria.

Immunohistochemistry for SMA, F4/80, Galectin-3, CD36 and iNOS can be estimated from each positive area as indication for the extent of inflammation and fibrosis.

### Bile Duct Models of Liver Fibrosis

These experiments are done to evaluate the efficacy of the compounds described herein on the fibrosis of the liver following bile duct ligation or treatment with drugs that cause biliary fibrosis. Animals treated with the compounds herein described show that liver fibrosis was reduced in comparison to vehicle controls.

### Example 9: In Vivo Animal Models of Lung Fibrosis

These experiments are done to evaluate the efficacy of the compounds described herein on the prevention of bleomycin-induced pulmonary fibrosis. An untreated control group with intratracheal saline infusion consists of between 6 and 12 mice. Bleomycin is administered by slow intratracheal infusion into the lungs of other groups on Day 0. On Days −1, 2, 6, 9, 13, 16 and 20, mice are dosed (iv, ip, subcut, or oral) once daily with vehicle or various doses of compounds described herein (iv, ip, subcut, or oral). Animals are weighed and evaluated for respiratory distress daily. On Day 21, all animals are euthanized and the wet weight of lungs is measured. Upon sacrifice, blood is collected via retro-orbital bleed for preparation of serum. The right lobe of the lung is snap frozen for subsequent hydroxyproline analysis while the left is insufflated and fixed in 10% formalin for histological analysis. The formalin-fixed lung is processed for routine histological evaluation.

### Example 10: In Vivo Animal Models of Kidney Fibrosis

These experiments are done to evaluate the efficacy of the compounds described herein on the fibrosis of the kidney using models of unilateral ureteral ligation and diabetic nephropathy. Animals treated with various compounds herein show that kidney fibrosis is reduced in comparison to vehicle controls.

### Example 11: In Vivo Animal Models of Cardiovascular Fibrosis

These experiments are done to evaluate the efficacy of the compounds described herein on the fibrosis of the heart and vessels using models of heart failure, atrial fibrillation, pulmonary hypertension, and atherosclerosis. Animals treated with various compounds herein show that cardiovascular fibrosis was reduced in comparison to vehicle controls.

### Example 12: VEGF-A-Induced Angiogenesis

Vascular endothelial growth factors (VEGFs) signaling though VEGF receptor-2 (VEGFR-2) is the primary angiogenic pathway. Galectin proteins are important for the signaling pathway. Compounds described herein are able to inhibit neovascularization of mouse cornea in response to injury.

### Example 13: Evaluation of Compound Absorption, Distribution, Metabolism, and Elimination

Compounds described herein are evaluated for physicochemical properties, including but not limited to solubility (Thermodynamic and Kinetic method), various pH changes, solubility in biorelevant medium (FaSSIF, FaSSGF, FeSSIF), Log D (Octanol/water and Cyclohexane/water), chemical stability in plasma, and blood partitioning.

Compounds described herein are evaluated for in vitro permeability properties, including but not limited to PAMPA (parallel artificial membrane permeability assay), Caco-2, and MDCK (wild type)

Compounds described herein are evaluated for animal pharmacokinetic properties, including but not limited to pharmacokinetics by various routes viz., oral, intravenous, intraperitoneal, subcutaneous in mice (Swiss Albino, C57, Balb/C), rats (Wistar, Sprague Dawley), rabbits (New Zealand white), dogs (Beagle), Cynomolgus monkeys, etc., tissue distribution, brain to plasma ratio, biliary excretion, and mass balance.

Compounds described herein are evaluated for protein binding, including but not limited to plasma protein binding (ultra Filtration and Equilibrium Dialysis) and microsomal protein binding.

Compounds described herein are evaluated for in vitro metabolism, including but not limited to cytochrome P450 inhibition, cytochrome P450 time dependent inhibition, metabolic stability, liver microsome metabolism, S-9 fraction metabolism, effect on cryopreserved hepatocyte, plasma stability, and GSH trapping.

Compounds described herein are evaluated for metabolite identification, including but not limited to identification in vitro (microsomes, S9 and hepatocytes) and in vivo samples.

### Example 14

The affinity of the tetrameric se-galactoside and trimeric Se-galactoside of Table 3 were assayed using the fluorescent polarization assay format of FIG. 6B.

FIG. 18D shows the expected affinity of the tetrameric Se-galactoside to Galectin-3. FIG. 18C shows the expected affinity of the trimeric Se-galactoside to Galectin-3.

Tetrameric Se-galactoside is expected to have higher affinity to the CRD versus the trimeric structure due to additional potential interaction of hydroxyl groups with aminoacids in the CRD vicinity.

### Example 15

As demonstrated in FIG. 5B, an ELISA format was developed that uses different pairs of galectins and integrins to investigate the cross reactivity of the compounds disclosed herein with galectins other than Gal-3, e.g. Galectin 1 and Galectin 9.

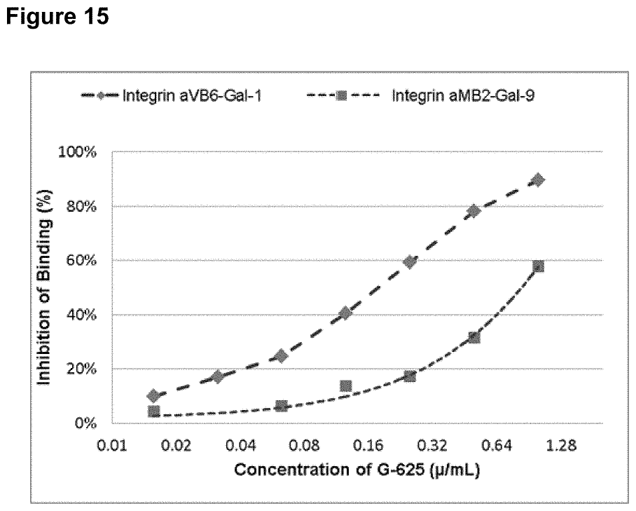

FIG. 15 shows that the compound G-625 significantly inhibited the interaction between Galectin 1 and integrin aBV6 as well as Galectin-9 and integrin aMB2. These data supports that the compounds disclosed herein can have selectivity to inhibit galectins other than Galectin-3. Such Galectins have been reported to be involved in a number of pathological pathways.

### Example 17—G-625 Synthesis Procedure

The G-625 compound was synthesized using the following scheme (see FIG. 4)

Step-1:

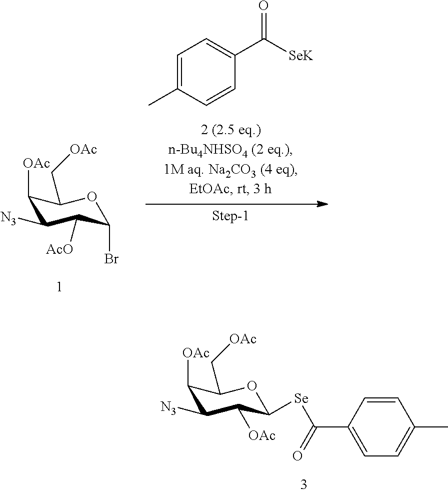

(2R,3R,4S,5R,6S)-2-(acetoxymethyl)-4-azido-6-((4-methylbenzoyl)selanyl) tetrahydro-2H-pyran-3,5-diyl diacetate (3): To a solution of (2R,3R,4S,5R,6R)-2-(acetoxymethyl)-4-azido-6-bromotetrahydro-2H-pyran-3,5-diyl diacetate (1, 1.6 g, 4.06 mmol) and potassium 4-methylbenzoselenoate (2, 2.41 g, 10.14 mmol) in EtOAc (30 mL), tetra-n-butyl ammonium hydrogen sulphate (2.75 g, 8.12 mmol) and aq. Na₂CO₃ (16 mL, 16 mmol) were added sequentially at room temperature (rt) and the reaction mixture was stirred at room temperature for 3 h. After completion, the reaction mixture was quenched with water (30 mL) and extracted with EtOAc (3×15 mL). The combined organic layers were dried (Na₂SO₄), filtered and concentrated in vacuo and the residue was purified by flash column chromatography [normal phase, silica gel (100-200 mesh), gradient 0 to 30% EtOAc in hexane] to afford the title compound (3) as a white solid (1.38 g, 66%).

¹H-NMR (400 MHz; CDCl₃): δ2.04 (s, 3H), 2.06 (s, 3H), 2.18 (s, 3H), 2.45 (s, 3H), 2.76-2.80 (m, 1H), 4.03-4.17 (m, 3H), 5.44-5.53 (m, 3H), 7.27 (d, J=8.1 Hz, 2H), 7.75 (d, J=8.1 Hz, 2H).

Step-2:

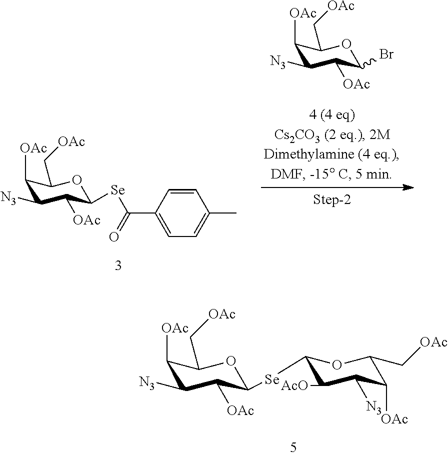

(2S,2′S,3R,3′R,4S,4′S,5R,5′R,6R,6′R)-selenobis(6-(acetoxymethyl)-4-azido tetrahydro-2H-pyran-2,3,5-triyl) tetraacetate (5): A solution of (2R,3R,4S,5R,6S)-2-(acetoxymethyl)-4-azido-6-((4-methyl benzoyl)selanyl) tetrahydro-2H-pyran-3,5-diyl diacetate (3, 100 mg, 0.19 mmol) in DMF (4 mL) was degassed with argon for 20 min. The mixture was cooled to −15° C. and Cs₂CO₃ (127 mg, 0.79 mmol), dimethylamine (2M in THF) (0.39 mL, 0.78 mmol) and a solution of (2R,3R,4S,5R)-2-(acetoxymethyl)-4-azido-6-bromotetrahydro-2H-pyran-3,5-diyl diacetate (307 mg, 0.78 mmol) in DMF (2 mL) were added and again degassed with argon for 20 min. The reaction mixture was stirred at same temperature for 5 min. After checking TLC, the reaction mixture was quenched with water (10 mL) and extracted with EtOAc (3×15 mL). The combined organic layers were washed with brine, dried (Na₂SO₄), filtered and concentrated in vacuo. The crude residue was purified by flash column chromatography [normal phase, silica gel (100-200 mesh), gradient 0% to 50% EtOAc in hexane] to afford the title compound (5) as colorless sticky solid (66 mg, 48%).

MS: m/z 707 (M+AcOH)⁺(ES⁺)

¹H-NMR (crude) (400 MHz; CDCl₃): δ2.04-2.19 (m, 18H), 2.87-2.98 (m, 2H), 4.09-4.17 (m, 6H), 4.60-4.82 (m, 6H).

Step-3:

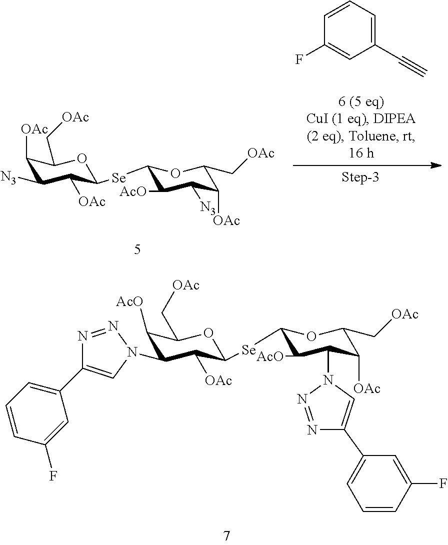

(2S,2′S,3R,3′R,4S,4′S,5R,5′R,6R,6′R)-selenobis(6-(acetoxymethyl)-4-(4-(3-fluoro phenyl)-1H-1,2,3-triazol-1-yl)tetrahydro-2H-pyran-2,3,5-triyl) tetraacetate (7): To a solution of (2S,2′S,3R,3′R,4S,4′S, 5R,5′R,6R,6′R)-selenobis(6-(acetoxymethyl)-4-azidotetrahydro-2H-pyran-2,3,5-triyl) tetraacetate (5, 130 mg 0.183 mmol) and 1-ethynyl-3-fluorobenzene (6, 115 mg, 0.918 mmol) in toluene (4 mL), DIPEA (0.07 mL, 0.366 mmol) and CuI (34 mg, 0.183 mmol) were added at room temperature. The reaction mixture was stirred at room temperature for 16 h. After completion, the reaction mixture was quenched with water (20 mL) and extracted with EtOAc (3×15 mL). The combined organic layers were filtered through a pad of celite bed, washed with EtOAc, dried (Na₂SO₄) and concentrated in vacuo and the residue was washed with Et₂O (10 mL) to afford the title compound (7) as a white solid (164 mg, 94%).

MS: m/z 949 (M+H)⁺(ES⁺)

¹H-NMR (400 MHz; DMSO-d₆): δ1.83 (s, 3H), 1.85 (s, 3H), 1.90-2.07 (m, 12H), 4.07-4.13 (m, 4H), 4.32-4.40 (m, 2H), 5.36 (d, J=9.5 Hz, 1H), 5.48-5.49 (m, 3H), 5.64-5.73 (m, 4H), 7.18 (t, J=8.4 Hz, 2H), 7.47-7.51 (m, 2H), 7.68-7.74 (m, 4H), 8.76 (d, J=10.3 Hz, 2H).

Step-4:

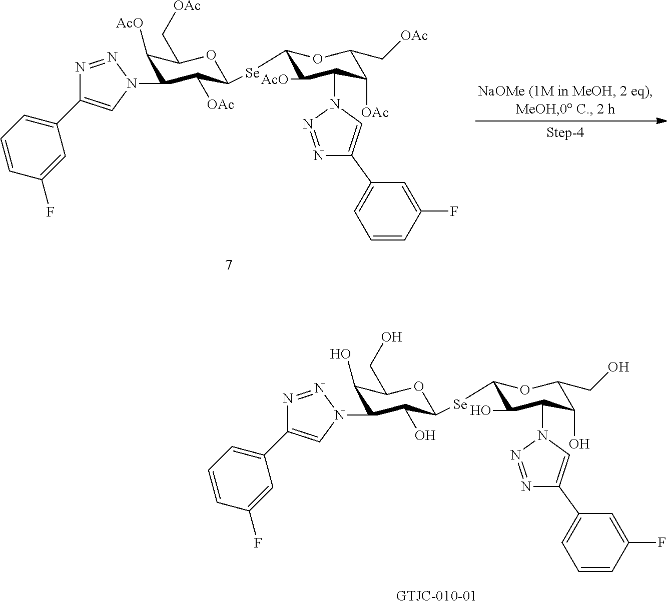

(2R,2′R,3R,3′R,4S,4′S,5R,5′R,6S,6′S)-6,6′-selenobis(4-(4-(3-fluorophenyl)-1H-1,2,3-triazol-1-yl)-2 -(hydroxymethyl)tetrahydro-2H-pyran-3,5-diol) (GTJC-010-01): To a solution of (2S,2′S,3R,3′R,4S,4′S,5R,5′R,6R,6′R)-selenobis(6-(acetoxymethyl)-4-(4-(3-fluorophenyl)-1 H-1,2,3-triazol-1-yl)tetrahydro-2H-pyran-2,3,5-triyl) tetra acetate (7, 200 mg, 0.21 mmol) in MeOH (10 mL), NaOMe (0.4 mL, 0.42 mmol) was added at 0° C. The reaction mixture was stirred at 0° C. for 2 h. After completion, the reaction mixture was acidified with Amberlyst 15H (pH ˜6), filtered, washed with MeOH and concentrated in vacuo. The crude residue was purified by prep-HPLC (reverse phase, X BRIDGE Shield RP, C-18, 19×250 mm, 5 μ, gradient 50% to 82% ACN in water containing 5 Mm Ammonium bicarbonate, 214 nm, RT: 7.8 min to afford the title compound as a white solid (GTJC-010-01, 18 mg).

LCMS (Method A): m/z 697 (M+H)⁺(ES⁺), at 4.51 min, purity 96%.

¹H-NMR (400 MHz; DMSO-d₆): δ3.49-3.61 (m, 4H), 3.72 (t, J=6.2 Hz, 2H), 3.99 (dd, 2.9 & 6.6 Hz, 2H), 4.36-4.43 (m, 2H), 4.70 (t, J=5.5 Hz, 1H), 4.82 (dd, 2.8 & 10.5 Hz, 2H), 5.19 (d, J=9.7 Hz, 2H), 5.31 (d, J=7.2 Hz, 2H), 5.40 (d, J=6.6 Hz, 2H), 7.12-7.17 (m, 2H), 7.46-7.51 (m, 2H), 7.66 (dd, J=2.3 & 10.2 Hz, 2H), 7.72 (d, J=7.8 Hz, 2H), 8.67 (s, 2H).

LCMS (Method A): Instruments: Waters Acquity UPLC, Waters 3100 PDA Detector, SQD; Column: Acquity BEH C-18, 1.7 micron, 2.1×100 mm; Gradient [time (min)/solvent B in A (%)]: 0.00/2, 2.00/2, 7.00/50, 8.50/80, 9.50/2, 10.0/2; Solvents: solvent A=5 mM ammonium acetate in water; solvent B=acetonitrile; Injection volume 1 μL; Detection wavelength 214 nm; Column temperature 30° C.; Flow rate 0.3 mL per min.

1. A compound of Forn a (5) or a pharmaceutically acceptable salt or solvate thereof

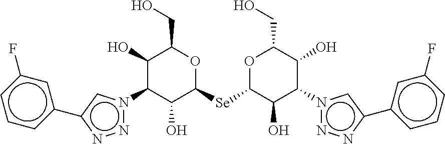

2. The compound of claim 1, wherein the compound has a binding affinity for galectins.

3. The compound of claim 1, wherein the compound has from at least about 2 to at least about 3 times higher affinity to galectin-3 compared to a corresponding thiogalactoside compound.

4. A composition comprising:
a therapeutically effective amount of a compound of Formula (5) 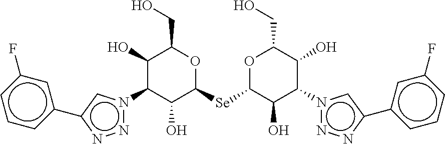
or a pharmaceutically acceptable salt or solvate thereof; and
a pharmaceutically acceptable adjuvant, excipient, formulation carrier or a combination thereof.

5. The composition of claim 4 further comprising a synergistic active agent.

6. A method for treating a disorder relating to the binding of a galectin to a ligand, comprising administering to a subject in need thereof a therapeutically effective amount of a compound of Formula (5) 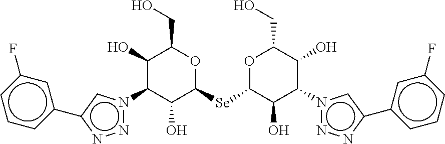
or a pharmaceutically acceptable salt or solvate thereof.

7. The method of according to claim 6, wherein the disorder is selected from the group consisting of inflammatory disorder, fibrosis, cancer, autoimmune diseases, metabolic disorders.

8. The method of claim 6, wherein the disorder is fibrosis and the fibrosis is selected from the group consisting of pulmonary fibrosis, liver fibrosis, kidney fibrosis and fibrosis of the heart.

9. The method of claim 6, wherein the disorder is atherosclerosis or pulmonary hypertension.

10. The method of claim 6, wherein the disorder is heart failure, arrhythmias, or uremic cardiomyopathy.

11. The method of claim 6, wherein the disorder is arthritis, rheumatoid arthritis, asthma, systemic lupus erythematosus or inflammatory bowel disease.

12. The method of claim 6, comprising administering the compound or the pharmaceutically acceptable salt or solvate thereof in combination with an anti-neoplastic drug.

13. The method of claim 12, wherein the anti-neoplastic drug is a checkpoint inhibitor, an immune modifier, an anti-neoplastic agent or a combination thereof.

14. The method of claim 13, wherein the checkpoint inhibitor is an anti-CTLA2, an anti-PD1, and anti-PDL1 or a combination thereof.

15. The method of claim 6, comprising administering parenterally the compound or the pharmaceutically acceptable salt or solvate thereof.

16. The method of claim 6, comprising administering enterally the compound or the pharmaceutically acceptable salt or solvate thereof.

17. The method of claim 6, comprising administering topically the compound or the pharmaceutically acceptable salt or solvate thereof.
